(19) United States
(12) Patent Application Publication
(10) Pub. No.: US 2010/0137258 A1
(43) Pub. Date: Jun. 3, 2010

(54) THIOPHOSPHI(O)NIC ACID DERIVATIVES AND THEIR THERAPEUTICAL APPLICATIONS

(76) Inventors: Francine Acher, Vaucresson (FR); Chelliah Selvam, Tamil nadu (IN); Jean-Philippe Pin, Montpellier (FR)

Correspondence Address: NIXON & VANDERHYE, PC, 901 NORTH GLEBE ROAD, 11TH FLOOR, ARLINGTON, VA, 22203, US

(21) Appl. No.: 12/452,988

(22) PCT Filed: Aug. 1, 2008
(86) PCT No.: PCT/IB2008/002990

§ 371 Date: Feb. 1, 2010

Related U.S. Application Data
(60) Provisional application No. 60/935,219, filed on Aug. 1, 2007.

(51) Int. Cl. A61K 31/662; C07F 9/30; C07F 9/32; A61P 25/00; A61P 25/22

(52) U.S. Cl. 514/121; 562/9; 558/172; 558/132; 514/120

(57) ABSTRACT

The invention relates to thiophosphi(o)nic acid derivatives having formula (I) wherein. M is a [C(R₃,R₄)]n1-C(E,COOR₁,N(H,Z)) group, or an optionally substituted Ar—CH(COOR₁,N(H,Z)) group (Ar designating an aryl or an heteroaryl group), or an α, β cyclic aminoacid group such as, formula (II) or a β, &ggr;-cyclic aminoacid group such as, formula (III). R₁ is H or R, R being an hydroxy or a carboxy protecting group, such as C₁-C₃ alkyl, Ar (being aryl or heteroaryl), Z is H or an amino protecting group R′, such as C₁-C₃ alkyl, C₁-C₃ acyl, Boc, Fmoc, COOR, benzyl oxycarbonyl, benzyl or benzyl substituted such as defined with respect to Ar; E is H or a C1-C3 alkyl, aryl, an hydrophobic group such as (CH₂)ₙ₁-alkyl, (CH₂)ₙ₁-aryl (or heteroaryl), such as a benzyl group, or a xanthyl, alkyl xanthyl or alkyl thioxanthyl group, or —(CH₂)ₙ₁-cycloalkyl, —(CH₂)ₙ—(CH₂—Ar)₂, a chromanyl group, particularly 4-methyl chromanyle, indanyle, tetrahydro naphtyl, particularly methyl-tetrahydronaphtyl; or M is OM′, wherein M′ is as above defined for M; R₂ is selected in the group comprising: D-CH(R₆)—C—(R₇,R₈)—(R₁₁,R₁₂)CH—C(R₉.R₁₀)-D-CH(OH)-D-[C(R₁₃,R₁₄)]ₙ₃—C[(R₁₅,R₁₆,R₁₇]ₙ₄-D-CH₂—(R₁₈)CH═C(R₁₉)-D-(M₁)n6-CO-D-C(R,R′)—O-D-O—, formula (IV), PO(OH)₂—CH₂ or (PO(OH)₂—CH₂), (COOH—CH₂)—CH₂— with -D=H, OH, OR, (CH₂)ₙ₂OH, (CH₂)ₙ₁OR, COOH, COOR, (CH₂)ₙ₂COOH, (CH₂)ₙ₁C00R, SR, S(OR), SO₂R, NO₂, heteroaryl, C₁-C₃ alkyl, cycloalkyl, heterocycloalkyl, (CH₂)ₙ₂-alkyl, (COOH, NH₂)—(CH₂)ᵤ₁-cyclopropyl-(CH₂)ᵤ₂—, CO—NH-alkyl, Ar, (CH₂)ₙ₂—Ar, CO—NH—Ar, R being as above defined and Ar being an optionally substituted aryl or heteroaryl group, —R₃ to R₁₉, identical or different, being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)XPO₃H₂, with x=0, 1 or 2, B(OH)₂, formula (V), NO₂, SO₂NH₂, SO₂NHR; SR, S(O)R, SO₂R, benzyl; one of R₁₁ or R₁₂ being COOR, COOH, (CH₂)ₙ₂—COOH, (CH₂)ₙ₂—COOR, PO₃H₂ the other one being such as defined for R₉ and R₁₀;—one Of R₁₅, R₁₆ and R₁₇ is COON or COOR, the others, identical or different, being such as above defined;—one of R₁₈ and R₁₉ is COOH or COOR, the other being such as above defined;—M₁ is an alkylene or arylene group;—n1=1, 2 or 3;—n2=1, 2 or 3,—n3=0, 1, 2 or 3 and—n4=1, 2 or 3;—n5=1, 2 or 3;—n6=0 or 1,—u1 and u2, identical or different=0, 1 or 2, Ar, and alkyl groups being optionally substituted by one or several substituents on a same position or on different positions, said substituents being selected in the group comprising: OH, OR, (CH₂)ₙ₁OH, (CH2)ₙ₁OR, COOH, COOR, (CH₂)ₙ₁C00H, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂, formula (V), NO₂, SO₂NH₂, SO₂NHR; SR, S(O)R, SO₂R, benzyl; R being such as above defined.

The invention relates to thiophosphi(o)nic acid derivatives having agonist or antagonist properties for metabotropic glutamate receptors (mGluRs), in particular agonist or antagonist properties for group III, subtype 4, metabotropic glutamate receptors (mGlu4Rs) and their therapeutical applications.

MGluRs are of particular interest in medicinal chemistry because they are believed to be suitable targets for treating a large variety of brain disorders such as convulsions, pain, drug addiction, anxiety disorders, and several neurodegenerative diseases.

The eight known subtypes of mGluRs are classified into three groups. Group III contains subtypes 4 and 6-8. Mainly located presynaptically, where they act as autoreceptors, group III mGluRs decrease adenylyl cyclase activity via a G_{1/10} protein and are specifically activated by L-AP4. Among this group, mGlu4R is thought to be a possible new target for Parkinson's disease, but the lack of a highly specific agonist has seriously impaired target validation studies. Furthermore, despite many chemical variations around the structure of glutamate, L-AP4 remains the strongest mGlu4R agonist with an EC₅₀ of only 0.32 μM and its α-methyl analogue, a competitive antagonist with an IC₅₀ of 100 μm. New chemotypes of higher potency and specificity are to be found.

The inventors' researches in that field lead them to develop methods of synthesis of thiophosphi(o)nic acids making it possible to obtain a large number of valuable agonists or antagonists for mGlu4Rs, and valuable antagonists corresponding to the α-substituted derivatives thereof.

An object of the invention is then to provide new thiophosphi(o)nic acid derivatives, particularly having agonist or antagonist properties for group III mGluRs.

Another object of the invention is to provide new methods of synthesis of biologically active thiophosphi(o)nic acid derivatives with a large variety of substituents.

According to still another object, the invention takes advantage of the mGlu4Rs agonists or antagonist properties of the thiophosphi(o)nic acid derivatives thus obtained and aims to provide pharmaceutical compositions useful for treating brain disorders.

The thiophosphi(o)nic acid derivatives of the invention are diasteroisomers or enantiomers of formula (I)

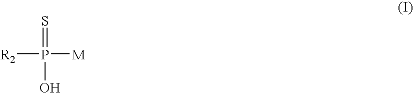

wherein
M is a [C(R₃,R₄)]ₙ₁—C,(E,COOR₁,N(H,Z)) group, or an optionally substituted Ar—CE,(COOR₁,N(H,Z)) group (Ar designating an aryl or an heteroaryl group), or an α, β cyclic aminoacid group such as,

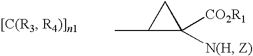

or a β,γ-cyclic aminoacid group such as

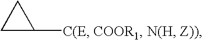

R₁ is H or R, R being an hydroxy or a carboxy protecting group, such as C₁-C₃ alkyl, Ar (being aryl or heteroaryl),

Z is H or an amino protecting group R′, such as C₁-C₃ alkyl, C₁-C₃ acyl, Boc, Fmoc, COOR, benzyl oxycarbonyl, benzyl or benzyl substituted such as defined with respect to Ar;

E is H or a C₁-C₃ alkyl, aryl, an hydrophobic group such as (CH₂)ₙ₁-alkyl, (CH₂)ₙ₁-aryl (or heteroaryl), such as a benzyl group, or a xanthyl, alkyl xanthyl or alkyl thioxanthyl group, or —(CH₂)ₙ₁-cycloalkyl, —(CH₂)ₙ—(CH₂—Ar)₂, a chromanyl group, particularly 4-methyl chromanyle, indanyle, tetrahydro naphtyl, particularly methyl-tetrahydronaphtyl;

or
M is O-M′, wherein M′ is as above defined for M;
R₂ is selected in the group comprising:

### D-CH(R₆)—C—(R₇,R₈)—

### (R₁₁,R₁₂)CH—C(R₉,R₁₀)—

### D-CH(OH)—

D-[C(R₁₃,R₁₄)]ₙ₃—
—C[(R₁₅,R₁₆,R₁₇)]ₙ₄—

### -D-CH₂—

### (R₁₈)CH═C(R₁₉)—

D-(M₁)ₙ₆-CO—

### -D-C(R,R′)—O—

### -D-O—

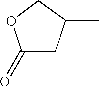

### PO(OH)₂—CH₂ or (PO(OH)₂—CH₂), (COOH—CH₂)—CH₂—

with

D=H, OH, OR, (CH₂)ₙ₂OH, (CH₂)ₙ₁OR, COOH, COOR, (CH₂)ₙ₂COOH, (CH₂)ₙ₁COOR, SR, S(OR), SO₂R, NO₂, heteroaryl, C₁-C₃ alkyl, cycloalkyl, heterocycloalkyl, (CH₂)ₙ₂-alkyl, (COOH,NH₂)—(CH₂)ᵤ₁-cyclopropyl-(CH₂)ᵤ₂—, CO—NH-alkyl, Ar, (CH₂)ₙ₂—Ar, CO—NH—Ar, R being as above defined and Ar being an optionally substituted aryl or heteroaryl group,

R₃ to R₁₉, identical or different, being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂,

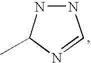

NO₂, SO₂NH₂, SO₂NHR; SR, S(O)R, SO₂R, benzyl;
one of R₁₁ or R₁₂ being COOR, COOH, (CH₂)n₂—COOH, (CH₂)n₂—COOR, PO₃H₂ the other one being such as defined for R₉ and R₁₀;

one of R₁₅, R₁₆ and R₁₇ is COOH or COOR, the others, identical or different, being such as above defined;

one of R₁₈ and R₁₉ is COOH or COOR, the other being such as above defined;

M₁ is an alkylene or arylene group;

n1=1, 2 or 3;

n2=1, 2 or 3,

n3=0, 1, 2 or 3 and

n4=1, 2 or 3;

n5=1, 2 or 3;

n6=0 or 1,

u1 and u2, identical or different=0, 1 or 2,

Ar, and alkyl groups being optionally substituted by one or several substituents on a same position or on different positions, said substituents being selected in the group comprising: OH, OR, (CH₂)ₙ₁OH, (CH₂)ₙ₁OR, COOH, COOR, (CH₂)ₙ₁COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂

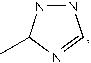

NO₂, SO₂NH₂, SO₂NHR; SR, S(O)R, SO₂R, benzyl; R being such as above defined.

In the above defined thiophosphi(o)nic acid derivatives of the invention, D is preferably Ar (optionally substituted), Ar—(CH₂)ₙ₂ (with Ar optionally substituted), C₁-C₃ alkyl or cycloalkyl; alkyl —(CH₂)ₙ₂, or COOH. Preferably Ar is a phenyl group (optionally substituted) or a carboxyalkyl group (optionally substituted). Alternatively, Ar is an heterocyclic group (optionally substituted). Advantageous groups are thiophenyl or furanyl group (optionally substituted).

A first preferred family corresponds to thiophosphi(o)nic acid derivatives of formula (II)

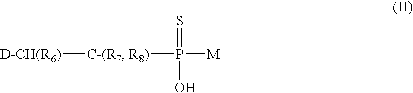

wherein the substituents are as above defined.

In particularly preferred derivatives of this family, D is Ar or a substituted Ar, especially a phenyl group optionally substituted by 1 to 5 substituents. The substituents are in ortho and/or meta and/or para positions. Preferred substituents comprise: OH, OR, (CH₂)ₙ₂OH, (CH₂)ₙ₂OR, COOH, COOR, (CH₂)ₙ₂COOH, (CH₂)ₙ₂COOR, C1-C3 alkyl or cycloalkyl, (CH₂)ₙ₂-alkyl, aryl, (CH₂)ₙ₂-aryl, halogen, CF₃, SO₃H, PO₃H₂, B(OH)₂ alkylamino, fluorescent group (dansyl, benzoyl dinitro 3, 5′,

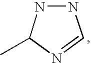

NO₂, SO₂NH₂, SO₂(NH,R)SR, S(O)R, SO₂R, OCF₃, heterocycle, heteroaryl, substituted such as above defined with respect to Ar. Advantageously, R₆ and/or R₇ and/or R₈ are H, C₁-C3 alkyl, OH, CF₃, NH₂.

A second preferred family corresponds to thiophosphi(o)nic acid derivatives of formula (III)

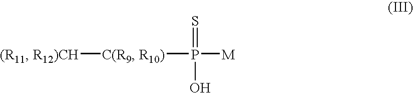

wherein the substituents are as above defined.

In preferred derivatives, one of R₁₁ or R₁₂ is COOH.

Advantageously, the other one of R₁₁ or R₁₂, and/or R₉ and/or R₁₀ are H, C₁-C₃ alkyl, OH, NH₂, CF₃.

A third preferred family corresponds to thiophosphi(o)nic acid derivatives of formula (IV)

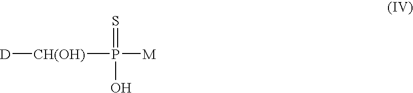

wherein the substituents are as above defined.

In preferred derivatives, D is as above defined with respect to formula (II)

In a fourth preferred family, the thiophosphi(o)nic acid derivatives have formula (V)

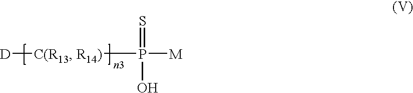

wherein the substituents are as above defined, one of R₁₃ or R₁₄ representing OH.

In preferred derivatives, D is as above defined with respect to formula (II).

The substituent R₁₃ or R₁₄ which does not represent OH is advantageously H, C₁-C₃ alkyl, OH, CF₃, NH₂.

In a fifth preferred family, the thiophosphi(o)nic acid derivatives have formula (VI)

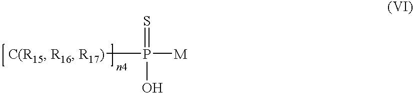

wherein the substituents are as above defined.

In preferred derivatives, in the first group of the chain, one or two of R₁₅, R₁₆ or R₁₇ are COOH, the other(s) advantageously being H, C₁-C₃ alkyl, OH, NH₂, CF₃.

In a sixth family, the thiophosphi(o)nic acid derivatives have formula (VII)

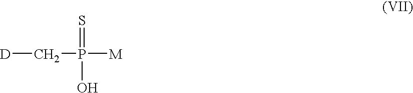

wherein the substituents are as above defined.

In preferred derivatives, D is as above defined with respect to formula (II).

In a seventh family, the thiophosphi(o)nic acid derivatives have formula (VIII)

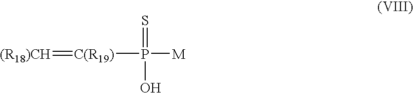

wherein the substituents are as above defined.

In preferred derivatives, R₁₈ is COOH.

Advantageously, R₁₉ is H, C₁-C₃ alkyl, OH.

An eighth family corresponds to thiophosphi(o)nic acid derivatives of formula (LIX)

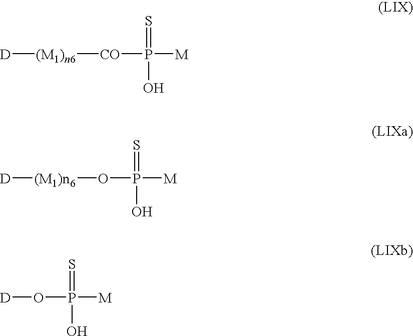

wherein the substituents are as above defined.

In preferred derivatives, either n6=0, or n6=1 and M, is an alkylene or arylene group such as above defined.

In a preferred embodiment of the invention, M is a [C(R₃,R₄)]ₙ₁—C(E,COOR₁,N(H,Z)) group, in the above defined hypophosphorous acid derivatives.

Preferably R₃ and/or R₄ are H and n1=1 or 2, more preferably 2.

In another preferred embodiment, M is an Ar group or a substituted arylene group, particularly a C₆H₄ group or a substituted C₆H₄ group, the substituents being as above defined with respect to formula I.

In still another embodiment, M comprises a cyclic aminoacid group, particularly, M is an α, β cyclic aminoacid group such as

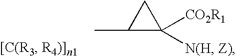

or a β,γ-cyclic aminoacid group such as

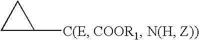

The invention particularly relates to the above mentioned derivatives wherein E represents H, which are group III mGluR agonists, and more particularly mGlu4R agonists of great interest.

The invention also particularly relates to the above mentioned derivatives wherein E is different from H and is more especially a C₁-C₃, alkyl, an aryl, an hydrophobic group such as a (CH₂)ₙ₁-alkyl group, or a (CH₂)ₙ₁-aryl group, as above defined, particularly a benzyl group, or a methylxanthyl group or alkylxanthyl or alkylthioxanthyl.

Advantageously, such derivatives are valuable mGluR antagonists, particularly mGlu4 antagonists.

The invention also relates to a process for preparing thiophosphi(o)nic acid derivatives of formula I

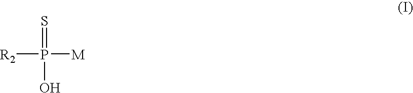

wherein the substituents are as above defined.

According to method A), said process comprises

a1) treating a derivative of formula (IX)

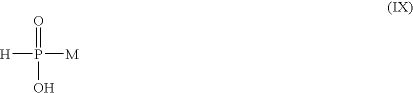

wherein the substituents and n1 are as above defined,
with either trimethylsilylchloride (TMSCl) and triethylamine (Et3N), or N,O-(bis-triethylsilyl)acetamide (BSA), (Et representing a C₂H₅ group).
a2) adding to the reaction product
one of the following derivatives having, respectively,

D-C(R₆)═C(R₇,R₈), or formula X

(R₁₁,R₁₂)C═C(R₉,R₁₀) formula XI

formula XII:

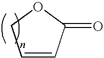

D-CH(═O) formula XIII

D-[C(R₁₃,R₁₄)]ₙ₃—Br formula XIV

[C(R₁₅,R₁₆,R₁₇)]ₙ₄—Br formula XV

D-I formula XVI

(R₁₈)C≡C(R₁₉) formula XVII

a3) replacing the P═O moiety by P═S moiety, by protecting the hydroxymethyl group when present and the phosphi(O)nic acid before introducing the sulphur atom by the use of the Laweson's reagent or PSCl₃,
a4) performing hydrolysis in two steps, comprising 1) LiOH or KOH hydrolysis of esters; 2) deprotection under acid conditions at 60-80° C.,
a5) treating the reaction product under acidic conditions or with catalysts to obtain the final desired product;
a6) recovering the diastereoisomers or the enantiomer forms,
a7) if desired, separating diastereoisomers, when obtained, into the enantiomers.

According to method B, said process comprises

b1) treating a derivative of formula (XVIII)

(R″SiO)₂—P—H (XVIII)

wherein R″ is a C₁-C₃ alkyl

with

either a derivative of formula (X)

D-C(R₆)═C(R₇,R₈) (X)

or with a derivative of formula (XI)

(R₁₁,R₁₂)C═C(R₉,R₁₀) (XI)

wherein one of R₉ or R₁₀ is COOalk, alk being a C₁-C₃ alkyl
b2) treating the condensation product with a dibromo derivative of formula (XIX)

Br—[C(R₃,R₄)]ₙ₁—Br (XIX)

under reflux conditions; and adding HC(Oalk)₃
wherein alk is a C1-C3 alkyl
b3) treating the condensation product with a derivative of formula (XX)

NH(Z)—CH(CO₂R)₂ (XX)

in the presence of K₂CO₃, BuO₄NBr, under reflux conditions;
b4) replacing the P═O moiety by P═S moiety, by protecting the hydroxymethyl group when present and the phosphi(O)nic acid before introducing the sulphur atom by the use of the Laweson's reagent or PSCl₃,
b5) performing hydrolysis in two steps, comprising 1) LiOH or KOH hydrolysis of esters; 2) deprotection under acid conditions at 60-80° C.,
b6) treating the condensation product under acidic conditions or with catalyst to obtain the final desired product;
b7) recovering the diastereoisomers or the enantiomer forms, and
b8) if desired, separating diastereoisomers, when obtained, into the enantiomers.

Alternatively, the reaction product obtained at step b1) is reacted according to step b21), with a derivative of formula (XXI)

[(R₃,R₄)C]ₙ₁═C(COOR₁,NH(Z)) (XXI)

In step b3i), the reaction product is treated under acidic conditions to give the final desired product.

According to method C, said process comprises

c1) reacting, as defined in step a1), a derivative of formula (XXII)

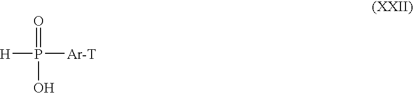

wherein Ar is as above defined and preferably an optionally substituted C₆H₄ group and T represents a C₁-C₃ alkyl group
c2) carrying out reaction step a2) by using one of the derivatives of formula (X) to (XVII)
c3) treating the reaction product with NBS, AIBN to have bromo derivatives with Ar substituted by T′-Br, with T′=CH₂
c4) reacting the bromo derivative thus obtained with (CH)₆N₄ in an organic solvent, then AcOH/H₂O to obtain cetone derivatives with Ar substituted by —C═O;
c5) treating the cetone derivatives with KCN, NH₄Cl and NH₄OH to obtain aminocyano derivatives, with Ar substituted by —C(CN,NH₂),
c6) replacing the P═O moiety by P═S moiety, by protecting the hydroxymethyl group when present and the phosphi(O)nic acid before introducing the sulphur atom by the use of the Laweson's reagent or PSCl₃,
c7) performing hydrolysis in two steps, comprising 1) LiOH or KOH hydrolysis of esters; 2) deprotection under acid conditions at 60-80° C.,
c8) treating under acidic conditions to obtain derivatives with Ar substituted by —C(COOR,NH₂), and
c9) treating with catalysts to obtain the final desired product.

Method D is used for the preparation of thiophosphonates of formula Iy

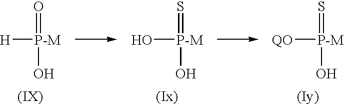

wherein Q is D-C(R,R′)— or D-
d1) treating a derivative of formula (IX) with N,O-(bis-triethylsilyl)acetamide (BSA) and sulfur powder
d2) hydrolysing with 1N HCl to afford thiophosphonate (Ix)
d3) reacting thiophosphonate (Ix) with EDC (N-(3-Dimethylaminopropyl)-N′-ethylcarbodiimide), alcohol QOH in DMF or reacting thiophosphonate (Ix) with SOCl₂ at 0° C. and then with 1 equivalent of alcohol QOH
d4) treating the reaction product under acidic conditions or with catalysts or as C7 to obtain the final desired product (Iy).

Method E is used for the preparation of thiophosphonates of formula (Iz)

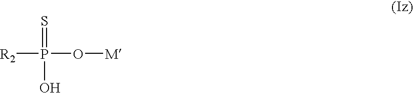

e1) Z-protected serine methyl ester O-phosphate (Z-Ser-(OMe)-O-phosphate) or homologues for example (homoserine) are treated as (Ix) in d3) (diesterification) and then as described in a3) and a4).
e2) or treating H₃PO₂ hypophorous acid as in b1) followed by reacting the condensation product as in d1) to afford (Iz) with M′=H, which is then reacted with Z-protected serine methyl ester (Z-Ser-(OMe)-OH) or homologues as in d3) and d4).

In method A, according to a preferred embodiment

the use of derivatives of formula (X)

D-CH(R₆)═C(R₇,R₈) (X)

with derivatives of formula (IX) results, in step a2), in intermediate derivatives of formula (XXIII)

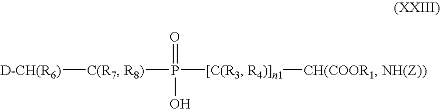

and, in step a5), in a final product of formula (XXIV)

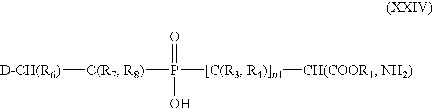

the use of derivatives of formula (XI) or formula (XII)

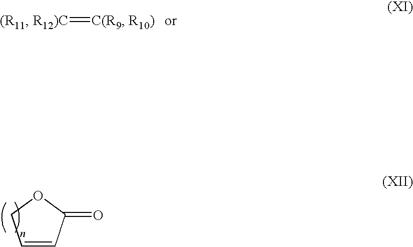

results, in step a2), in intermediate derivatives of formula (XXV)

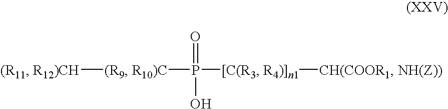

and, in step a5), in a final product of formula (XXVI)

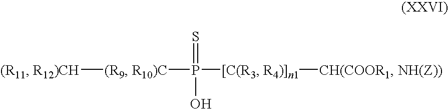

the use of derivatives of formula (XIII)

D-CH(═O) (XIII)

results, in step a2), in intermediate derivatives of formula (XXVII)

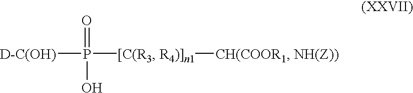

and, in step a5), in a final product of formula (XXVIII)

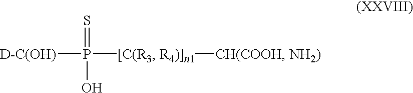

the use of derivatives of formula (XIV)

D-[C(R₁₃,R₁₄)]ₙ₃—Br (XIV)

results, in step a2), in intermediate derivatives of formula (XXIX)

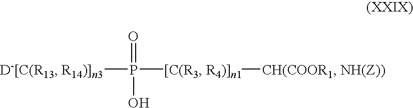

and, in step a5), in a final product of formula (XXX)

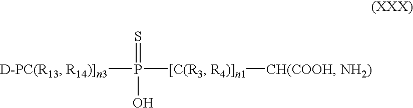

the use of derivatives of formula (XV)

[C(R₁₅,R₁₆,R₁₇)]ₙ₄—Br (XV)

results, in step a2), in intermediate derivatives of formula (XXXI)

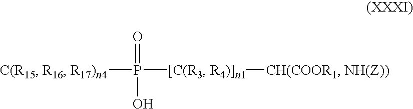

and, in step a5), in a final product of formula (XXXII)

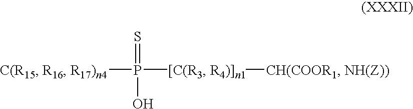

the use of derivatives of formula (XVI)

D-I (XVI)

results, in step a2), in intermediate derivatives of formula (XXXIII)

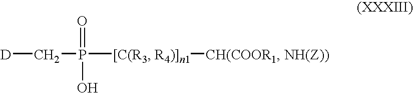

and, step a5), in a final product of formula (XXXIV)

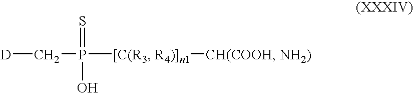

the use of derivatives of formula (XVII)

(R₁₈)C≡C(R₁₉) (XVII)

results, in step a2), in intermediate derivatives of formula (XXXV)

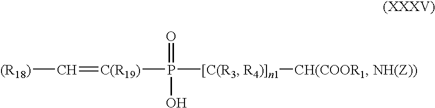

and, in step a5), in a final product of formula (XXXVI)

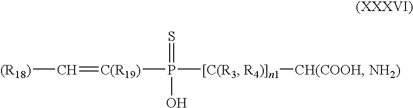

the use of derivatives of formula (LIX)

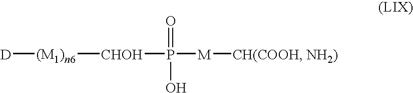

wherein M₁ is as above defined with respect to M and n6=0 or 1, and results by oxidation in a product of formula (LXI)

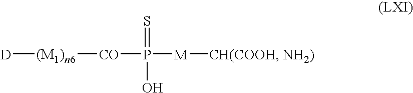

In method B,

the use, with derivatives of formula (XVIII),

(R″SiO)₂—P—H (XVIII)

of derivatives of formula (X)

D-CH(R₈)—C(R₇,R₈) (X)

results, in step b1), in intermediate derivatives of formula (XXXVII)

D-CH(R₆)—C(R₇,R₈)—P—(OSiR″)₂ (XXXVII)

in step b2), in intermediate derivatives of formula (XXXVIII)

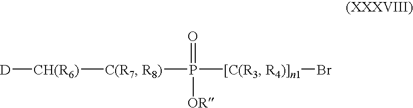

in step b5), in intermediate derivatives of formula (XXXIX)

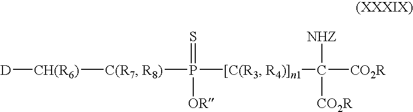

and, in step b6), in a final product of formula (XXXX)

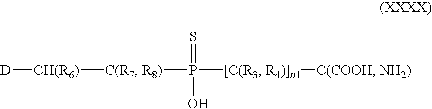

the use, with derivatives of formula (XVIII), of derivatives of formula (XI)

(R₁₁,R₁₂)C═C(R₉,R₁₀) (XI)

results, in step b1), in intermediate derivatives of formula (XXXXI)

(R₁₁,R₁₂)CH—C(R₉,R₁₀)—P—(OSiR″)₂ (XXXXI)

in step b2), in intermediate derivatives of formula (XXXXI)

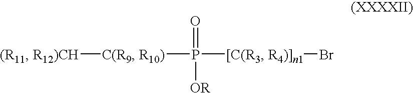

in step b5), in intermediate derivatives of formula (XXXXIII)

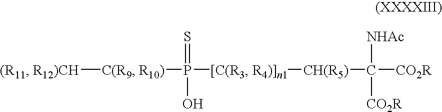

in step b6), in final products of formula (XXXXIV)

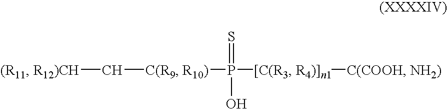

or, alternatively,

the use with derivatives of formula (XXXXI) obtained according to step b1)

(R₁₁,R₁₂)CH—C(R₉,R₁₀)—P—(OSiR″)₂ (XXXXI)

of derivatives of formula (XXXXV)

(R₃,R₄)C═C(COOR₁,NH(Z) (XXXXV)

results in intermediate derivatives of formula (XXXXVI)

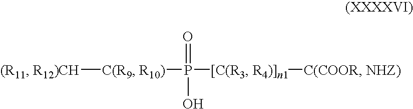

wherein the OH— group is then protected, the P═O moiety is replaced by a P═S moiety, the treatment under acidic conditions giving the final product of formula (XXXXVII)

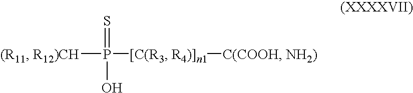

In method C,
the use, of a derivative of formula (XXII),

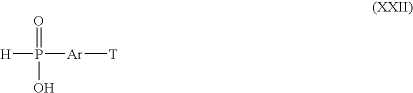

with a derivative of

D-C(R₆)═C(R₇,R₈), or formula X

(R₁₁,R₁₂)C═C(R₉,R₁₀) formula XI

formula XII:

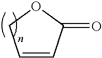

D-CH(═O) formula XIII

D-[C(R₁₃,R₁₄)]ₙ₃—Br formula XIV

[C(R₁₅,R₁₆,R₁₇)]ₙ₄—Br formula XV

D-I formula XVI

(R₁₈)C≡C(R₁₉) formula XVII

results in intermediate derivatives respectively having formulae (XXXXVIII) to (LIV)

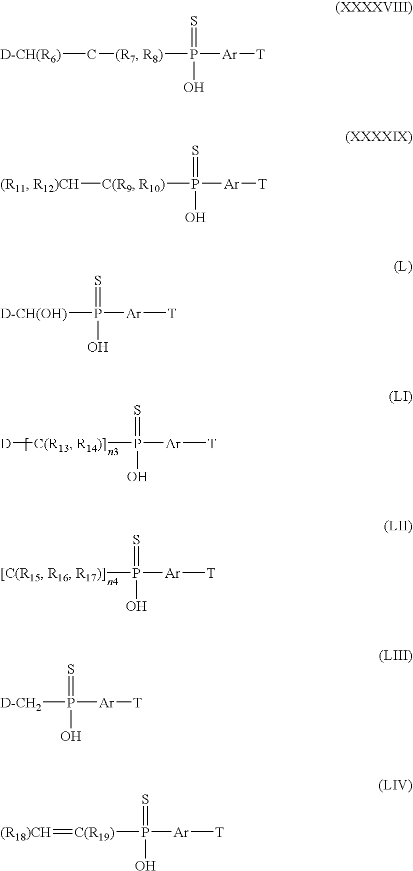

In method A, the derivatives of formula IX

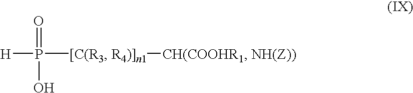

are advantageously obtained by reacting hypophosphorous acid of formula (LV)

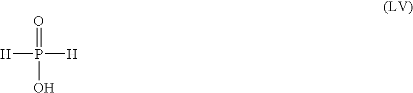

with a derivative of formula LVI

(R₃,R₄)ₙ₁C═CH—C(E,COOR₁,NH(Z)) (LVI)

Preferably, the derivative of formula (LVI) is Z-vinyl-glyOMe or a derivative thereof with E different from H, E being as above defined, and has formula (LVIa).

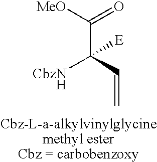

Z-vinyl-glyOMe is advantageously synthesized from methionine or glutamate according to references (1), (2) or (3).

Z-vinyl-glyOMe derivatives with E different from H can be prepared from α-alkyl methionine or alpha alkyl glutamate (see reference 4). Alpha amino acids can be stereoselectively α-alkylated using imidazolinones or oxazolidinones (references 5 and 6).

Other methods for obtaining Z-vinyl-glyOMe derivatives are given in Example 9.

The reaction is advantageously carried out in the presence of AIBN by heating above 50° C.-100° C., preferably at about 80° C.

In method B, the derivatives of formula (XVIII)

(R″SiO)₂—P—H (XVIII)

are advantageously obtained by reacting an hypophosphorous acid ammonium salt of formula (LVII)

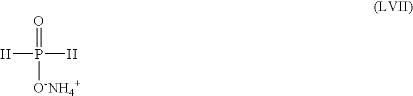

with a disilazane derivative of formula (LVIII)

(alk₃Si)₂—NH (LVIII)

The reaction is advantageously carried out under an inert gas, by heating above 100° C., particularly at about 120° C.,

or by reacting hypophosphorous acid with N,O-(bis-triethylsilyl)acetamide (BSA) at room temperature.

In method C, the derivatives of formula (XXII)

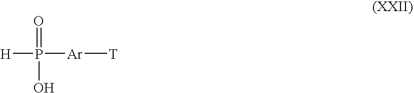

are advantageously obtained by reacting a mixture of H₃PO₂, Ar—NH₂, Ar—Br and a catalyst Pd(0) Ln. (Ln=n ligands).

The thiophosphi(o)nic acid derivatives which are intermediates in the above disclosed process, enter into the scope of the invention.

As above mentioned, said thiophosphi(o)nic acid derivatives have mGluRs agonist or antagonist properties of great interest and therefore are particularly valuable as active principles in pharmaceutical compositions to treat brain disorders.

They are particularly mGlu4Rs agonists or antagonists of great value.

The invention thus also relates to pharmaceutical compositions, comprising a therapeutically effective amount of at least one of the thiophosphi(o)nic acid derivatives of formula I in combination with a pharmaceutically acceptable carrier.

The invention also relates to the use of at least one of thiophosphi(o)nic acid derivatives of formula I for preparing a drug for treating brain disorders.

The pharmaceutical compositions and drugs of the invention are under a form suitable for an administration by the oral or injectable route.

For an administration by the oral route, compressed tablets, pills, capsules are particularly used. These compositions advantageously comprise 1 to 100 mg of active principle per dose unit, preferably 2.5 to 50 mg.

Other forms of administration include injectable solutions for the intravenous, subcutaneous or intramuscular route, formulated from sterile or sterilizable solution. They can also be suspensions or emulsions.

These injectable forms, for example, comprise 0.5 to 50 mg of active principle, preferably 1 to 30 mg per dose unit.

The pharmaceutical compositions of the invention prepared according to the invention are useful for treating convulsions, pain, drug addiction, anxiety disorders and neurodegenerative diseases.

By way of indication, the dosage which can be used for treating a patient in need thereof, for example, corresponds to doses of 10 to 100/mg/day, preferably 20 to 50 mg/day, administered in one or more doses.

The conditioning with respect to sale, in particular labelling and instructions for use, and advantageously packaging, are formulated as a function of the intended therapeutic use.

According to another object, the invention relates to a method for treating brain disorders, comprising administering to a patient in need thereof an effective amount of an thiophosphi(o)nic acid derivative such as above defined.

According to still another object, the invention relates to the use of at least one thiophosphi(o)nic acid derivative such as above defined for preparing a drug for treating drug disorders.

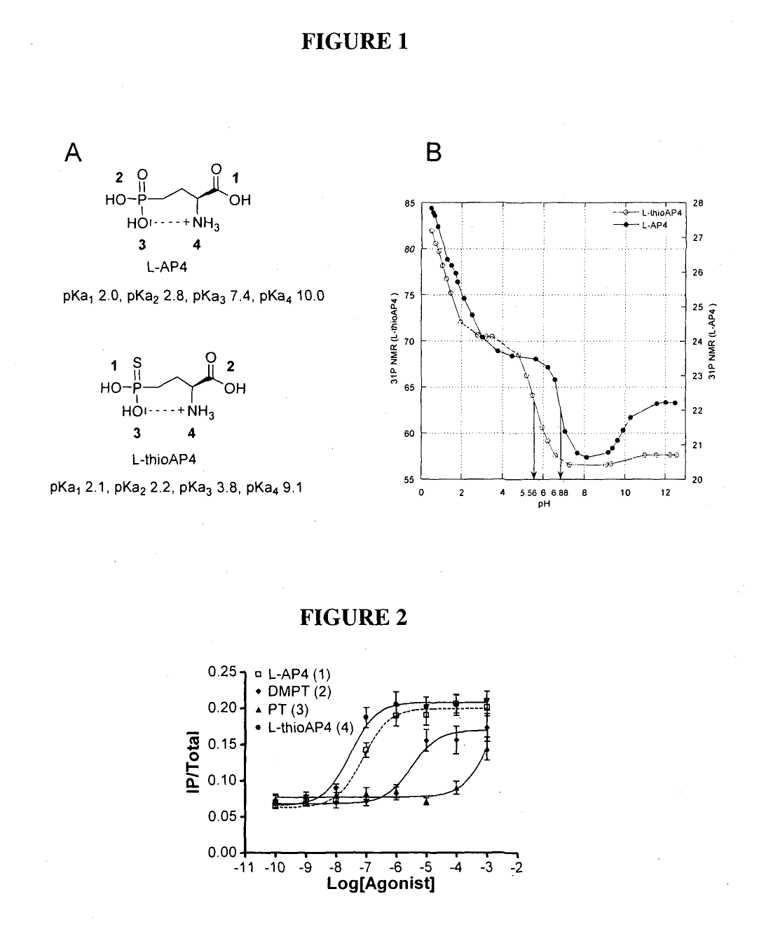

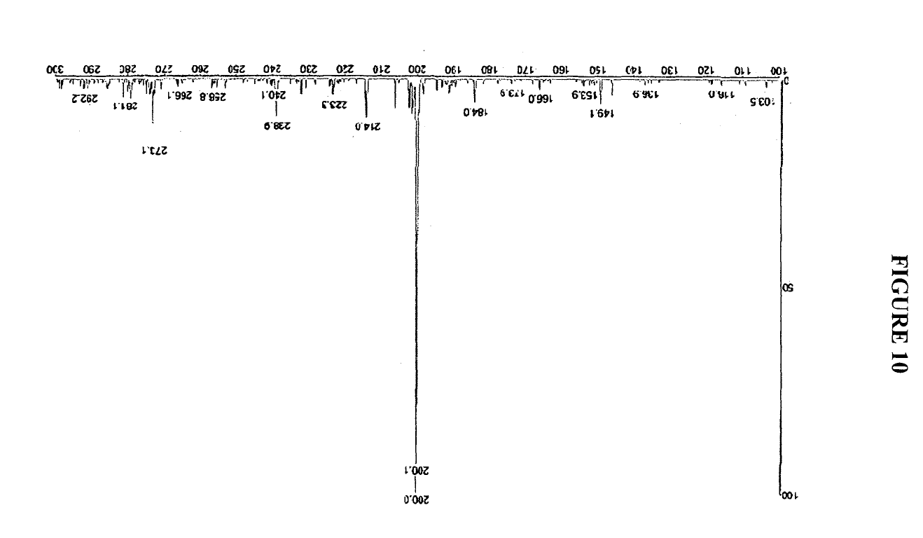

Other characteristics and advantages of the invention will be given in the following examples illustrating the synthesis of the thiophosphi(o)nic acid derivatives, wherein it is referred to FIGS. 1 to 10:

FIG. 1 gives the dissociation constants of L-AP4 and L-thio AP4;

FIG. 2, the dose response curves of L-AP4 and analogues on mGlu4 receptors;

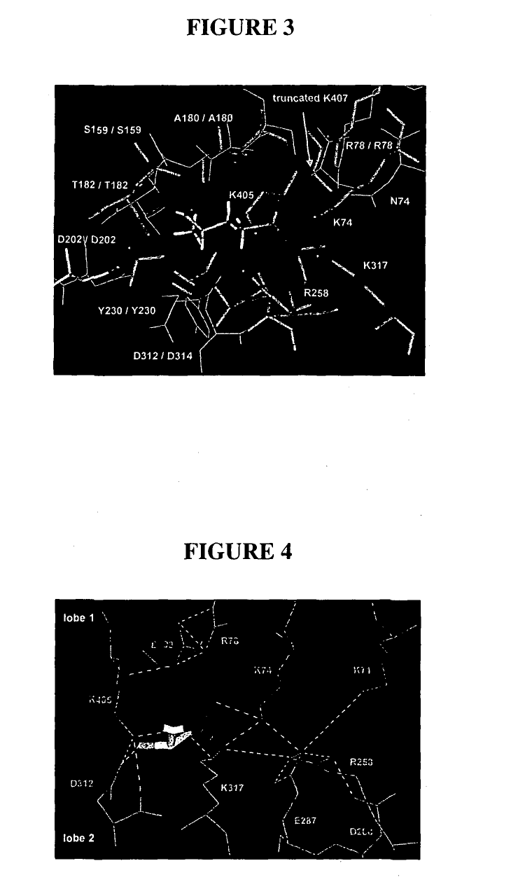

FIG. 3, the superimposition of Cₐ atoms of lobe 1 residues of mGlu7R (x-ray structure PDB cde 2e4z) and of mGlu4R docked with L-AP4 (1) (homology model);

FIG. 4, L-AP4 (1) docked at LBD;

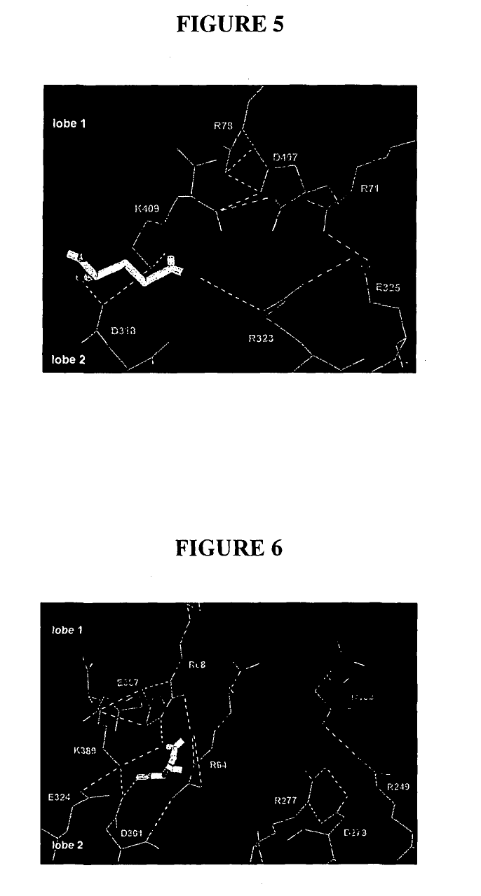

FIG. 5, the crystal structure of glutamate bound to the closed form of mGlu1R LBD (PDB code lewk:A);

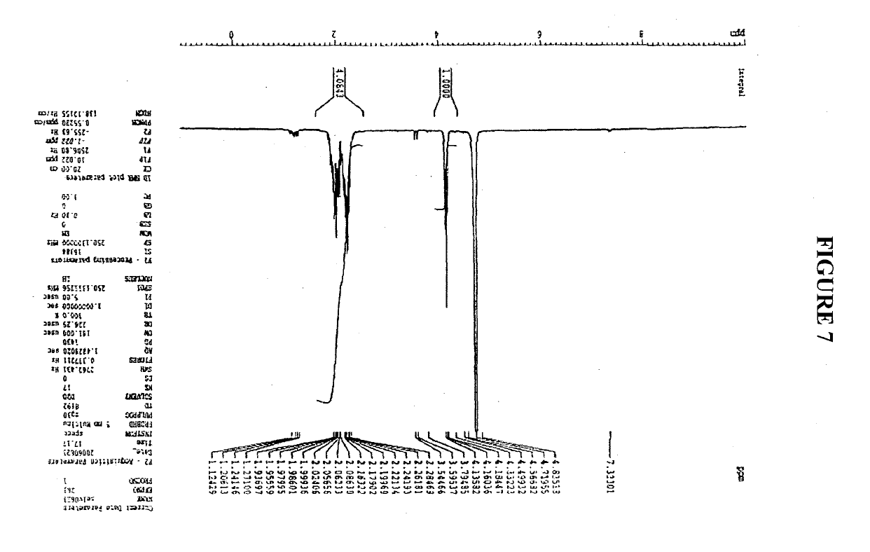

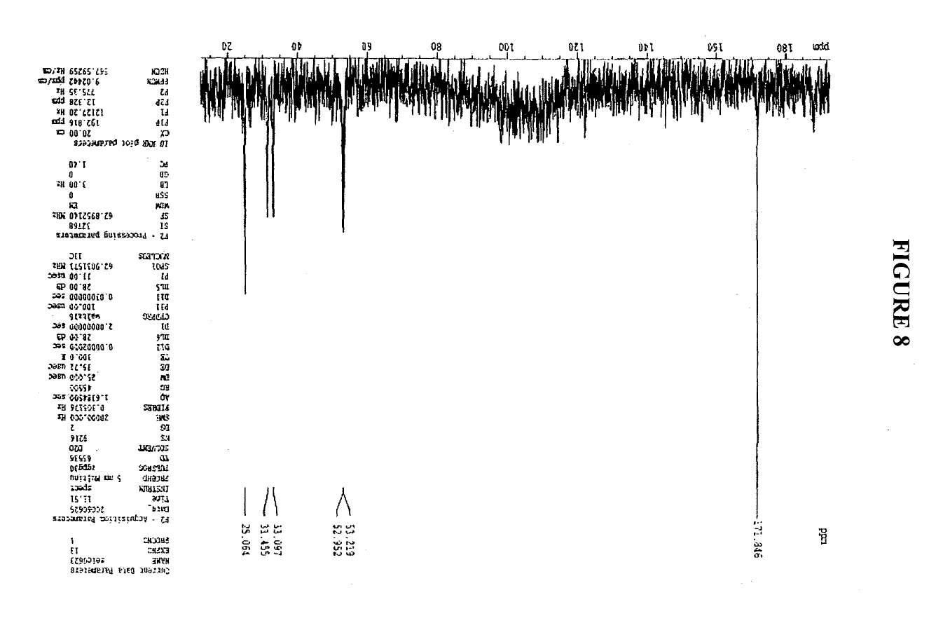

FIG. 6, the crystal structure of glutamate bound to the closed form of mGlu3R LBD (PDB code 2e4u);

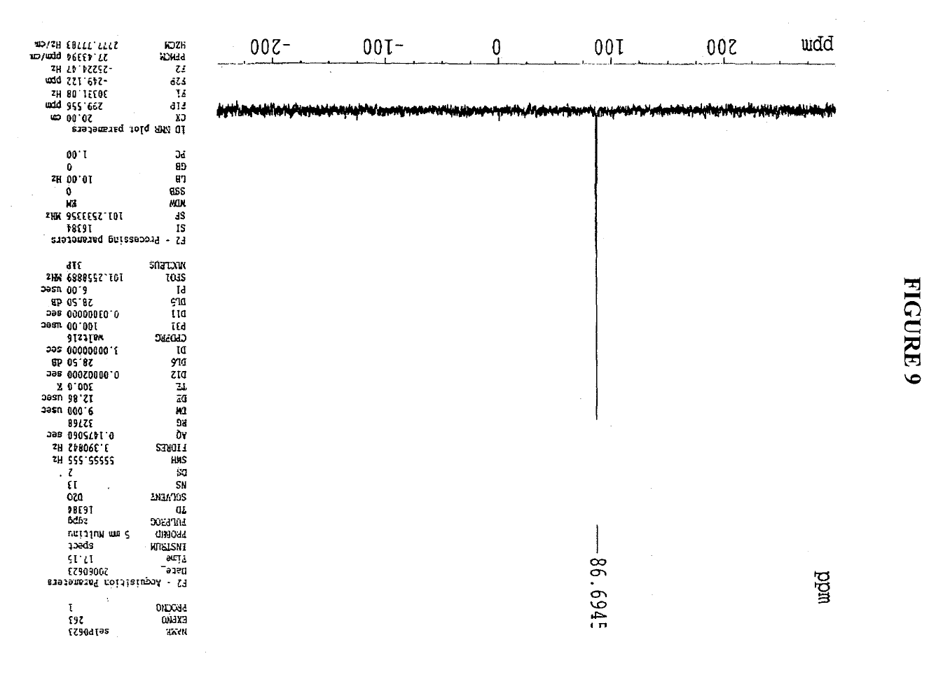

FIGS. 7-10, the ¹HMMR, ¹³CNMR, ³¹PNMR, mass spectra, respectively, of L-thioAP4;

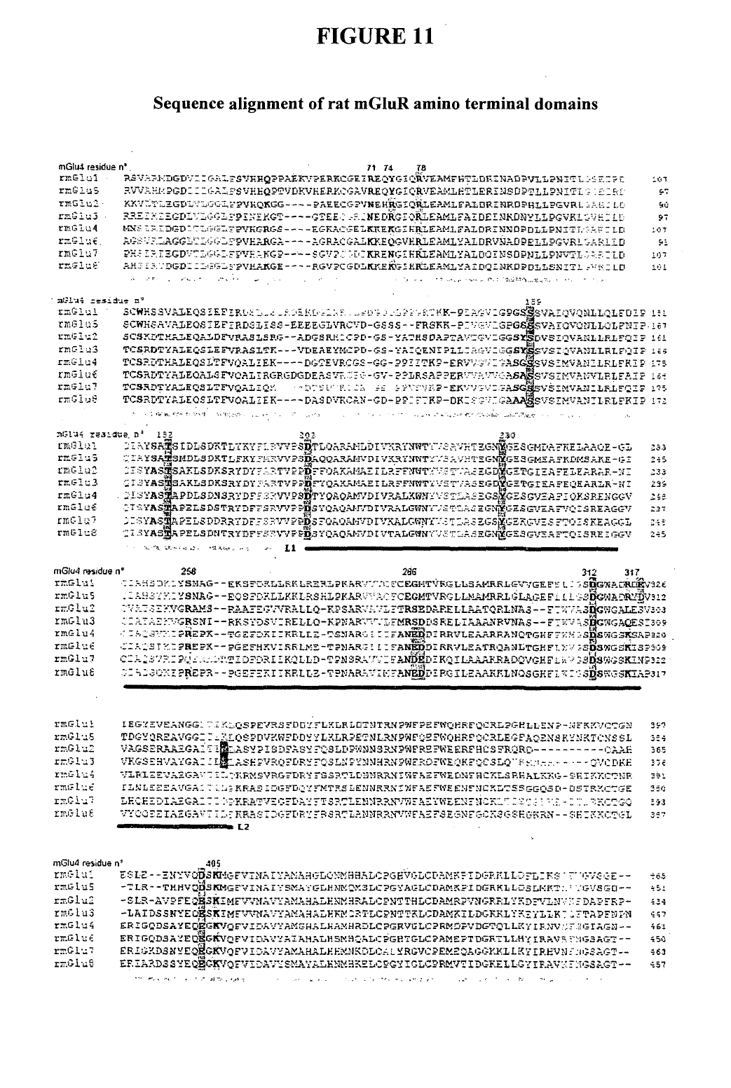

FIG. 11, the sequence alignment of rat mGluR amino terminal domains.

### RESULTS

### Chemistry

Results obtained with L-AP4 (2-amino-4-phosphonobutyric acid); L-thioAP4 (2-Amino-4-thiophosphonobutyric acid) tested as agonists of mGlu4, 6, 7, 8 receptors are given hereinafter:

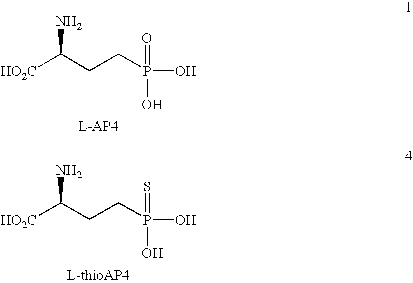

The γ-phosphinic acid derivative of glutamate 5 is a key intermediate in all the synthetic schemes. It was synthesized from aqueous hypophosphorous acid by a radical addition to the N-Z protected vinyl glycine methyl ester (Scheme 1 hereinafter).

The synthesis of H-phosphinic acid derivatives has been the subject of numerous studies in which the formation of the P—C bond usually results from the addition of a Phosphorous III moiety to unsaturated systems or activated halides. These reactions occur under base- or metal-catalyzed, or under radical conditions. When starting from hypophosphorous acid (H₃PO₂), the challenge is to limit the addition to one equivalent of substituent to obtain monosubstituted phosphinic acid. This problem is faced when bis(trimethylsilyloxy)phosphonite (BTSP) is used, where a large excess of BTSP (five equivalents) is required in order to yield only the H-phosphinic derivative. An alternative route has been suggested by Froestl et al. (ref. 1) for the synthesis of GABA phosphinic analogues. A temporary protection secured the monoalkylation, however yields were later reported to be low and non reproducible. The optimal reaction appears to occur under radical conditions. H-alkylphosphonates are cleanly obtained because their radical may not be formed for a second alkylation step.

The inventors choose to use α,α′-azoisobutyronitrile (AIBN) to initiate the radical condensation of H₃PO₂ to a protected vinylglycine to yield the protected H-phosphinic derivative 5.

Synthesis of the R-enantiomer of compound 5 has been reported by Zeng et al. (ref. 2) by the BTSP route and more recently a synthesis of the S-enantiomer in which Et₃B initiated radical addition of ammonium hypophosphite to Z-L-α-vinylGlyOMe has been described (ref. 3).

In view of the high cost of commercially available L-AP4, an alternate efficient synthetic route was developed starting from 5. Oxidation of the P—H bond of the protected phosphinic acid 5 was achieved by heating compound 5 with one equivalent of DMSO and a catalytic amount of iodine at 60° C. for 5 h to yield 6 (ref. 4). Deprotection of functional groups was performed by heating 6 in the presence of 6N HCl for 5 h and gave the desired product 1 in quantitative yield. Reactions are summarized in following scheme 1.

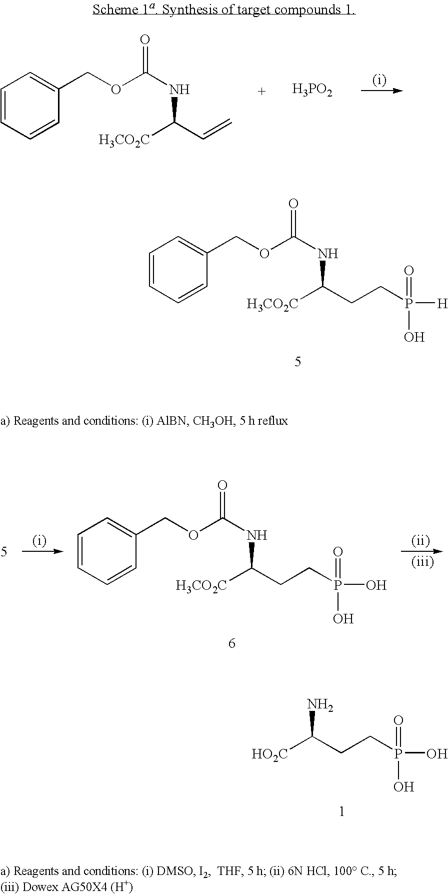

The protected H-phosphinic derivative 5 was oxidized to the corresponding thiophosphonate under mild conditions.

The synthesis of target compound 4 is given hereinafter:

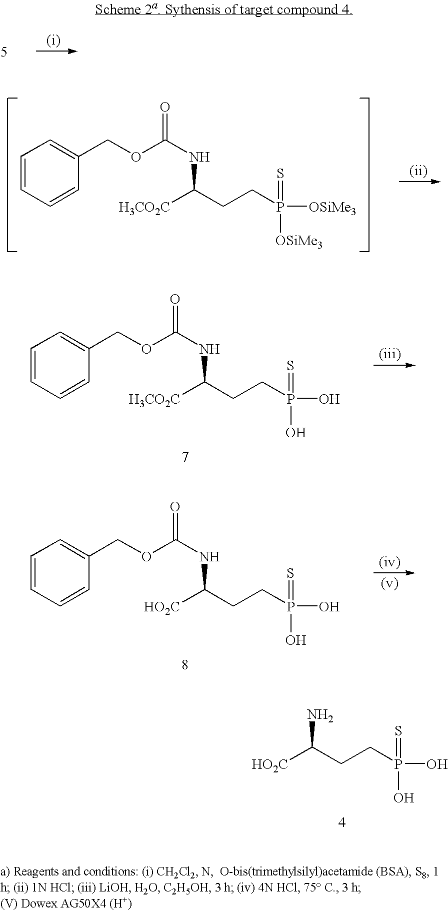

Compound 5 was silylated to the protected BTSP intermediate with N,O-bis(trimethylsilyl)acetamide (BSA). In the presence of sulfur powder this intermediate was smoothly oxidized to the protected thio phosphonate 7. Deprotection of 7 with 6N HCl at 90° C. for 3 h resulted in 35% of the desired product 4 and 65% of the hydrolyzed product 1 (ratio measured by ³¹P NMR). A milder deprotection was thus required. Hydrolysis of the carboxylic methyl ester of 7 was carried out with 3 equivalents of LiOH and afforded 8 in a good yield. Hydrogenolysis of 8 in the presence of palladium on charcoal failed to give 4. Thus, we turned to mild acidic deprotection of the benzyloxycarbonyl group. Treating 8 with 4N HCl at 75° C. for 3 h afforded the desired product 4 as the major product and 1 in smaller amount (7:3 ratio measured by P NMR). Compound 4 was purified by water elution on a cation exchange resin column. The purity of 4 was easily checked by P NMR because of a large difference in chemical shifts (86.7 ppm for 4 and 35.4 ppm for 1). This assessment was used to check the stability of 4 after several weeks of storage in aqueous solution at pH 7 at −20° C.

Because the potency of several group III mGluR agonists (e.g. ACPT-I (being aryl or heteroaryl), DCPG (being aryl or heteroaryl), L-AP4) is related to their additional acidic function, the inventors anticipated that the differences in pharmacological activities of L-AP4 1 and LthioAP4 4 would be due to their different ionization states. In order to compare them, the pKa values of these amino acids were evaluated. The results are illustrated by FIGS. 1A and 1B: A) Calculated pKa values using SPARC on-line calculator (ref. 5, 6). The ionizable functional groups 1 to 4 correspond to pKa₁, pKa₂, pKa₃ and pKa₄. B) Experimental pKa₃ values determined by plotting ³¹P NMR chemical shift versus pH variations for L-AP4 (1, blue) and L-thioAP4 (4, red). Indicated pKa₃ values were calculated by non-linear regression analysis using GraphPad Prism program. In addition, the ³¹P NMR chemical shifts of 1 and 4 are sensitive to the ionization state, thus titration curves may be obtained from their pH dependence (ref. 7-9) (FIG. 1B). L-AP4 1 and L-thioAP4 4 are characterized by four pKa values corresponding to the acidities of the α-carboxylic, the γ-phosphonate/thiophosphonate (pKa₁, pKa₂, pKa₃) and α-ammonium (pKa₄) groups. The pKa₁ and pKa₂ values are too close to be determined from the titration curves, however pKa₃ and pKa₄ are easily measured. Values of 6.88 and 9.90 were found for pKa₃ and pKa₄ of 1 and 5.56 and 9.70 for 4. It can be noted that shielding of the phosphorus atom (decrease of the ³¹P NMR chemical shift) is observed upon ionization of the three first acidic functions while a deshielding effect (increase of the ³¹P NMR chemical shift) occurs when the amine is deprotonated and its positive charge removed. Once the amine is deprotonated, this effect is suppressed and ³¹P NMR chemical shift increased because the electron density is then located more on the heteroatoms bound to the phosphorus atom. This effect is more pronounced with 1 than with 4 (FIG. 1B). Because pKa₁ and pKa₂ are well below 7, the corresponding functions (α-carboxylic acid and first acidity of the phosphonate/thiophosphonate group) are totally deprotonated at physiological pH. The second acidity of the phosphonate is only half deprotonated at that pH, because pKa₃ of 1 is found at 6.88. In contrast the same function is almost completely deprotonated in 4 because of the decrease of the pKa₃ value to 5.56. As a result the negative charge that bears the distal group of 4 is significantly increased in comparison to 1, and allows stronger interaction with the basic residues of the binding site.

Pharmacology

The effects of L-AP4 (1) and L-thioAP4 (4), were examined on all group III mGlu receptors (mGlu4, mGlu6, mGlu7 and mGlu8). The results are given in Table 1 below:

TABLE 1
Agonist activities of L-AP4 1 and thio analogue 4 at group III mGlu receptors.

| | mGlu4 | mGlu6 | mGlu7 | mGlu8 |
|---|---|---|---|---|
| agonists | EC50 (μM) | EC50 (μM) | EC50 (μM) | EC50 (μM) |
| L-AP4 (1) | 0.080 ± 0.017 (10) | 2.08 ± 0.38 (6) | 440 ± 120 (5) | 0.128 ± 0.019 (6) |
| L-thioAP4 (4) | 0.039 ± 0.006 (10) | 0.73 ± 0.06 (6) | 197 ± 55 (6) | 0.054 ± 0.080 (5) |

Data are the means ± SEM of (n) separate experiments.

These receptors were transiently expressed in HEK-293 cells as previously described. Since group III mGlu receptors are not naturally coupled to phospholipase-C but rather inhibit adenylyl cyclase, receptors were co-transfected with a chimeric G-protein alpha subunit which is recognized by these receptors but effectively activates the phospholipase-C pathway. Thus, the functional assay consisted in measuring the total inositol phosphate production resulting from receptor activation (ref. 10, 11).

L-AP4 analogue exhibited an agonist activity at group III mGlu receptors. L-thioAP4 (4) turned out to be about two fold more active than L-AP4 (1) on all subtypes (Student's paired T test: P<0.05). L-AP4 analogue displayed no selectivity among group III mGlu receptor subtypes.

Selectivity versus group I and II mGlu receptors was checked for L-AP4 analogues. At 100 μM, no agonist or antagonist activities were detected on receptors belonging to these groups, indicating that L-AP4 is selective group III mGlu receptor agonists.

Molecular Analysis

Molecular basis of the selectivity of 1 and 4 regarding group III mGlu receptors may be explained with the help of crystal structures (ref. 13, 14) and homology models (ref. 15) of the ligand binding domain (LBD). This domain folds in two lobes and adopts open or closed conformations. Agonists bind to lobe 1 in the open form of the LBD, and are then trapped in the closed form which was demonstrated to be required for receptor activation. The closed conformation is stabilized by agonist interactions with both lobes. It is assumed that the better that stabilization, the more potent the agonist is.

Herein, the inventors demonstrate that such an interpretation of the rank order of potency applies to the comparison of glutamate, L-AP4 (1), L-thio-AP4 (4) bound to the closed form of the LBD of mGlu receptors.

Because of a high receptor similarity among each of the three mGluR groups, one subtype of each group (mGlu1, mGlu3 and mGlu4) was chosen for the molecular analysis (see sequence alignment of rat mGluR amino terminal domains in FIG. 11). Thus the X-ray structures of glutamate bound to mGlu1 and mGlu3 LBD were used (Protein Data Bank codes: lewk:A and 2e4u) and a homology model of L-AP4 (1) bound to mGlu4 LBD. It was found that the crystal structure of mGlu7R bound to 2-(N-morpholino)ethanesulfonic acid (MES), a crystallization additive (PDB code: 2e4z), was not advantageous to building a new homology model of mGlu4 LBD because it displays an open conformation of the domain and a limited resolution. As a matter of fact, some side chains of residues likely contacting agonists are not solved (e.g. E405, K407, Q258 to R263) and MES does not bind to the signature motif that is found in all mGlu receptors. However the residues (Cα atoms) of lobe 1 in former 3D-model of mGlu4 docked with L-AP4 (1) was superimposed on those of the recent mGlu7R structure. The very good superimposition (rmsd 1.05 Å) attests of the accuracy of the model (FIG. 11). Moreover side chains of the binding site residues of lobe 1 and hinge are well oriented in the homology model in comparison to those of the X-ray structure (FIG. 3).

A close look at the binding residues around L-AP4 (1) reveals that five basic residues make ionic interactions with the distal phosphonate group. They are K74, R78 and K405 from lobe 1 and R258 and K317 from lobe 2 (FIG. 4). Among them, three of these basic residues (R78, K405 and R258) are simultaneously bound to acidic residues (E403, D312, E287, D288), so that their positive charge is neutralized. The two remaining basic residues (K74 and K317) are neutralized by the negative charges of the phosphonate group. With glutamate holding only one negative distal charge, the electrostatic stabilization is weaker resulting in a less potent agonist. A similar binding pattern as for L-AP4 (1) is found with L-thioAP4 (4) bound to mGlu4 receptor. Moreover, since the total charge of the thiophosphonate group is closer to two than with the L-AP4 (1) phosphonate group (see above), the electrostatic interaction of 4 to both lobes of the LBD is strengthened compared to 1. The increased stabilization of the closed conformation of the LBD bound to L-thioAP4 (4) may explain its higher potency at mGlu4 receptor binding site. The same interpretation may be suggested for the equally high potency of 4 at mGlu8 receptor since the binding pattern is the same at mGlu4 and mGlu8 binding sites. Interestingly the 1 and 4 EC₅₀ increase at mGlu6 and mGlu7 receptors (Table 1) correlates with a non optimal stabilization of the LBD in its closed conformation. Indeed K74 is replaced by a glutamine (Q58) in mGlu6 and by an aspargine (N74) in mGlu7 (FIG. 11), so that one of the ionic interaction is missing in comparison to 1/4 bound to mGlu4/8. In addition another basic residue analogous to R258 in mGlu4 receptor is missing in mGlu7 receptor (Q258) decreasing even more the stability of the active conformation of the LBD bound to 1 or 4. Nevertheless, in all group III mGlu receptors, the lysine of lobe 2 (K317 at mGlu4, K306 at mGlu6, K319 at mGlu7, K314 at mGlu8R) that makes a strong electrostatic interaction with 1 and 4 is conserved. Because this interaction is stronger with 4, potency of 4 is higher than that of 1 for all group III receptors.

The crystal structure of glutamate bound to mGlu1 receptor in the closed conformation of the LBD is given on FIG. 5. It shows that glutamate is bound to the conserved R78 and K409 of lobe 1 and to R323 from lobe 2, making a total of three basic residues. Each of these basic residues is also bound to an acidic residue (D407, D318, E325) so that the negative charge of the distal acidic group of glutamate does not seem to be mandatory. Indeed (S)3,5-dihydroxyphenylglycine, a well known agonist of group I mGlu receptors does not hold a distal charge. Yet the distal charge of glutamate allows a stronger interaction between K409 and glutamate in the open form of the LBD, in the first step of the activation process. L-AP4 (1) and L-thioAP4 (4) have no effect at mGlu1/5 receptors.

Crystal structure of glutamate bound to mGlu3 receptor in the closed conformation of the LBD shows that glutamate is bound to R68, K389 and R64 of lobe 1 and to none of the basic residues of lobe 2 (FIG. 6). Two of the three basic residues are bound to acidic residues (R68 to E387 and K389 to E324), consequently only one negative charge is needed on the agonist to afford a neutral system. A similar binding pattern may be expected for mGlu2 receptor according to the sequence alignments of FIG. 11. Accordingly the additional negative charges of L-AP4 (1) and L-thioAP4 (4) in comparison to glutamate do not afford any additional interactions with the protein. On the opposite, once the ligand is bound to the first lobe, the additional negative charge may then interact with some basic residues of lobe 2 that previously adopted a different conformation (e.g. R271 of mGlu2, R277 of mGlu3). The closing of the LBD may be modified preventing the activation of the receptor. As a matter of fact L-AP4 (1) was described as a modest mGlu2 receptor antagonist. It demonstrates that LAP4 (1) is able to bind to lobe 1 but that full closing of the LBD is hampered.

Several mutagenesis studies have been performed to better define the molecular determinants of the mGluR selectivity. They demonstrate that agonist selectivity derives from a set of distal residues that is specific to each group of mGlu receptors. The residues discussed in the present study are part of those sets. The geometry and the charge of the phosphonate and thiophosphonate groups of L-AP4 and L-thioAP4 fit best to the group III receptor cluster and not to those of group I/II receptors as explained above. This situation defines the molecular basis of the high potency and selectivity of L-AP4 and L-thioAP4 regarding the activation of group III mGlu receptors.

Discussion

Numerous preparations of L-AP4 have been published over decades, some used natural amino acids as starting material. The synthesis presented herein took advantage of the mild conditions of a radical condensation between vinylglycine and hypophosphorous acid.

Potency and selectivity of group III mGlu receptor agonists is brought by an additional acidic function which may be a carboxylic acid or the second acidity of a phosphonate group hold by glutamate analogues. Indeed this acidity seems to be critical as L-homocysteic acid, which is isosteric to L-AP4 (1) but with only one distal acidic moiety, shows no enhanced activity compared to glutamate at group III mGlu receptors. The purpose of the present invention was to further demonstrate the requirement for such an additional group for high activity.

L-thioAP4 (4) a sulfur analog of L-AP4 was synthesized and tested. L-thioAP4 (4) was able to activate group III mGlu receptors more efficiently than L-AP4 because of its stronger acidity as detailed below. To date, L-thioAP4 (4) is the most potent agonist described for these receptors.

L-thioAP4 (4) may exist in two tautomeric thiolo (P—SH) or thiono (P═S) forms in aqueous solution. Studies indicate that the thiono-thiolo equilibrium lies far on the thiono side in phosphonothioic acids. Sulfur makes much weaker hydrogen bonds than oxygen, thus the increased potency of L-thioAP4 may not result from such interactions. The particularity of the sulfur replacing the oxygen atom of the phosphoryl group (P═S replacing P═O) is that it has a major effect on the second acidity of the phosphonate. Calculated pKa values using SPARC on-line calculator for 1 distal group (pKa₁, pKa₃) are 2.8 and 7.4 while those of 4 (pKa₁, pKa₃) are 2.1 and 3.4. In contrast introducing withdrawing substituents in the 4 position of L-AP4 as in 4,4′-difluoro substituted L-AP4 affects both acidities as predicted pKas are 1.15 and 5.80. Dissociation of the second (thio)phosphonate acid group (pKa₃) is critical for the charge of this moiety at neutral pH. Experimental values were determined by ³¹P NMR titration to be 6.88 and 5.56 for 1 and 4 respectively. Thus at physiological pH, while L-AP4 (1) is only partially ionized, the thiophosphonate group of L-thioAP4 (4) is almost totally deprotonated and allows stronger electrostatic interaction with the five basic residues of the binding site that were identified in the 3D-model of L-AP4 docked at mGlu4 receptor binding site (FIG. 4). These interactions stabilize the active conformation of the binding domain which in turn triggers receptor activation. The superimposition of Cα atoms of lobe 1 residues of mGlu7R (X-ray structure PDB code 2e4z) and of mGlu4R docked with L-AP4 (1) (homology model) is given on FIG. 3. Residues 41-124, 150-202, 339-371 from mGlu4R and residues 41-124, 150-202, 341-373 from mGlu7 were superimposed with an rmsd of 1.05 Å. Most of the basic residues shown in FIG. 3 are conserved among the four subtypes of group III mGlu receptors, as a consequence L-thioAP4 (4) is not subtype selective. However, like L-AP4 (1), L-thioAP4 (4) is a group III selective agonist, displaying no agonist or antagonist activity on other groups of mGlu receptors. The potency and selectivity of L-AP4 (1) and L-thioAP4 (4) may be explained at the molecular level analyzing X-ray structures and homology models. While, the negative charges of their distal (thio)phosphonate group allow strong ionic interactions with the highly basic distal pocket of mGlu4/8 receptors, they allow no additional interactions at group I/II receptor binding sites in comparison with bound glutamate. In contrast, for these latter receptors, the extra charge may be deleterious as it may perturb the polar binding network around the ligand and prevent from reaching the active conformation of the LBD.

L-thioAP4 is more potent that L-AP4 and once radiolabelled, it becomes a useful pharmacological tool and allow the inventors to perform binding experiments that were limited up to now. Furthermore the structure-activity analysis of this compound disclosed new molecular features that will allow the design of more potent group III mGluR agonists which in turn may be developed as new drugs for psychiatric or neurodegenerative diseases and neuropathic pain relief.

In conclusion, the inventors have demonstrated that changing the phosphonate to a thiophosphonate (4) resulted in an increase of the activity. The enhanced potency of 4 is attributed to the increased second acidity of the thiophosphonate group and complete deprotonation of this group at physiological pH. Taken together these results confirm the critical role of the additional acidic function and its negative charge in glutamate analogues which are group III mGlu receptor agonists and led us to identify L-(+)-2-amino-4-thiophosphonobutyric acid 4 herein named L-thioAP4, as the most potent group III mGlu receptor agonist (EC₅₀=0.039, 0.73, 197, 0.054 μM at mGlu4, 6, 7, 8 respectively).

Experimental Section

Chemistry

All chemicals and solvents were purchased from commercial suppliers (Acros, Aldrich) and used as received. Glufosinate ammonium salt (PT 3) and Z-L-a-vinylGlyOMe (N-Benzyloxycarbonyl-a-vinylglycine methyl ester) were purchased from Riedel-de Haën (Sigma-Aldrich) and Ascent Scientific Ltd (North Somerset, UK) respectively. ¹H (250.13 MHz), ¹³C (62.9 MHz) and ³¹P (101.25 MHz) NMR spectra were recorded on an ARX 250 Bruker spectrometer. Chemical shifts (d, ppm) are given with reference to residual ¹H or ¹³C of deuterated solvents (CDCl₃ 7.24, 77.00; CD₃OD 3.30, 49.0; D₂O 4.80). For ³¹P NMR chemical shifts, SR values of −16664.43 Hz in D₂O and −15643.78 Hz in CD₃OD that were previously determined with an external reference (H₃PO₄ 95%), were used for calibration; the external reference was used for the titration experiments. Product visualization was achieved with 2% (w/v) ninhydrin in ethanol. Optical rotations were measured at the sodium D line (589 nm), at room temperature, with a Perkin-Elmer 341 polarimeter using a 0.1 or 1 dm pathlength cell. Mass spectra (MS) were recorded with a LCQ-advantage (ThermoFinnigan) mass spectrometer with positive (ESI+) or negative (ESI−) electrospray ionization, (ionization tension 4.5 kV, injection temperature 240° C.). Molecular models and 3D-structures were displayed using Discovery Studio 1.6 (Accelrys, San Diego).

Methyl (2S)-2-(N-benzyloxycarbonyl)amino-4-[(hydroxy)phosphinyl]butanoate (5). A mixture of hypophosphorous acid (H₃PO₂ 660 mg, 5 mmol, 50% aqueous), N-benzyloxycarbonyl-L-a-vinylglycine methyl ester (Z-L-α-vinylGlyOMe 249.3 mg, 1 mmol) and a,a′-azoisobutyronitrile (AIBN, 8.2 mg, 0.05 mmol) in methanol (1 mL) was refluxed at 80° C. for 5 h. Then the methanol was evaporated under vacuum and the residue was treated with 15 mL water and extracted with ethyl acetate (125 mL). The organic solution was washed with 10 mL of water, dried over anhydrous MgSO₄ and evaporated under vacuum to afford 5 (296 mg, 94% yield); ¹H NMR (CD₃OD): δ 1.98 (m, 4H), 3.72 (s, 3H), 4.11 (m, 1H), 5.12 (s, 2H), 7.08 (d, J_{PH}=565 Hz, 1H), 7.34 (m, 5H). ¹³C NMR (CD₃OD): δ 23.4, 26.1 (d, J=92 Hz), 52.2, 54.7, 66.9, 128.0, 128.3, 128.7, 137.2, 157.5, 172.7. P NMR (CD₃OD): δ 35.3.

(2S)-2-(N-Benzyloxycarbonyl)amino-4-phosphonobutyric Acid Methyl Ester (6). A mixture of 5 (0.90 mmol), DMSO (70 mg, 0.9 mmol) and iodine (1 mg) in THF 3 mL was stirred under heating at 60° C. for 5 h. The resulting mixture was evaporated to dryness under vacuum to give 6 (292 mg, 98% yield). ¹H NMR (CD₃OD): δ 1.94 (m, 4H), 3.72 (s, 3H), 4.31 (m, 1H), 5.12 (s, 2H), 7.35 (m, 5H). ³¹P NMR (CD₃OD): δ 29.61. ¹³C NMR (CD₃OD): 23.76 (d, J=139.79 Hz), 25.50, 52.16, 54.82 (d, J=18.56 Hz), 66.81, 127.99, 128.25, 128.71, 137.22, 157.53, 172.90.

L(+)-2-Amino-4-phosphonobutyric Acid (1). Compound 6 was dissolved in 5 mL of 6N HCl. The mixture was heated at 100° C. for 5 h and the resulting solution was cooled to room temperature. Volatile organic byproducts and water were removed under vacuum and the residue was purified using a Dowex AG50X4 cation exchange resin column (H⁺, 20-50 mesh, 24.1.7 cm, water elution). The fractions which gave positive color reaction with ninhydrin were combined and evaporated under vacuum to give 1 (quantitative yield). ¹H NMR (D₂O): δ 1.70 (m, 2H), 2.12 (m, 2H), 3.99 (t, J=6.02 Hz, 1H). ³¹P NMR (D₂O): 35.42. ¹³C NMR (D₂O): δ 23.61 (d, J=135.42 Hz), 24.68, 53.91 (d, J=13.37 Hz), 172.49. MS (ESI−) m/z 182.2 (M−1). [α]_{D}²⁰+13.2 (c 1.0, H₂O, lit.⁵⁵+10.3, c 2.0, H₂0).

Methyl (2S)-2-(N-benzyloxycarbonyl)amino-4-thiophosphonobutanoate (7). To a mixture of 5 (296 mg, 0.94 mmol) and sulphur powder (96 mg, 3 mmol) in 2 mL of methylene chloride at 0° C. under an argon atmosphere was added dropwise N,O-bis(trimethylsilyl)acetamide (BSA) (814 mg, 4 mmol). The mixture was allowed to warm to room temperature and stirred for 1 h, then cooled to 0° C. and 15 mL of 1N HCl were added, then extracted with ethyl acetate (2×100 mL). The combined organic solution was dried over anhydrous MgSO₄ and concentrated in vacuo (302 mg, 93%). ¹H NMR (CD₃OD): δ 2.10 (m, 4H), 3.78 (s, 3H), 4.32 (m, 1H), 5.11 (s, 2H), 7.33 (m, 5H). ³¹P NMR (CD₃OD): δ87.23. ¹³C NMR (CD₃OD): δ 26.21, 32.46 (d, J=108.38 Hz), 52.04, 54.51, 66.83, 127.87, 128.13, 128.60, 137.11, 157.58, 173.08.

(2S)-2-(N-Benzyloxycarbonyl)amino-4-thiophosphonobutyric Acid (8). To a solution of 7 (302 mg, 0.87 mmol) in ethanol (5 mL) at room temperature was added LiOH.H₂O (126 mg, 3 mmol) in ethanol: water (10+10 mL). The reaction was stirred at the same temperature for 3 h. Then 15 mL of 1N HCl was added and extracted with ethyl acetate (2.75 mL). The organic extracts were combined, dried over anhydrous MgSO₄, and concentrated under vacuum to give 8 (257 mg, 89% yield). ¹H NMR (CD₃OD): δ 1.91 (m, 4H), 4.03 (m, 1H), 5.12 (s, 2H), 7.36 (m, 5H). ³¹P NMR (CD₃OD): δ 87.36. ¹³C NMR (CD₃OD): δ 26.31, 32.51 (d, J=108.38 Hz), 54.51 (d, J=18.12 Hz), 66.80, 127.82, 128.09, 128.56, 137.11, 157.68, 174.30.

L(+)-2-Amino-4-thiophosphonobutyric acid (4). The crude compound 8 (147 mg, 0.44 mmol) was treated with 4 mL of 4N HCl and stirred at 75° C. for 3 h. The reaction mixture was concentrated under vacuum and the residue was purified using a Dowex AG50X4 cation exchange resin column (H⁺, 20-50 mesh, 24.1.7 cm, water elution, 12 mL fractions, ninhydrin product visualization). Collection of fractions 4-20 gave 53 mg of 4 containing 20% of L-AP4 1. This mixture was reloaded on the same column and eluted with water.

Fractions 4-9 afforded pure 4 (19 mg, 0.095 mmol, 22% yield), after evaporation. ¹H NMR (D₂O): δ 1.97 (m, 2H), 2.24 (m, 2H), 4.16 (t, J=6.03 Hz, 1H). ³¹P NMR (D₂O): δ 86.69. ¹³C NMR (D₂O): δ 25.06, 32.28 (d, J=103.28 Hz), 53.08, 171.85. MS (ESI+) m/z 200.0 (M+1). [α]_{D}²⁰+17 (c 1.0, H₂O).

pKa determination of L-AP4 (1) and L-thioAP4 (4). Each ³¹P NMR spectrum was acquired at 27° C. with external H₃PO₄ (95%) reference at 0 ppm (sealed capillary). Compound 1 (12 mg) or 4 (6.4 mg) was dissolved in 0.54 mL of H₂O and 0.06 mL of D₂O. The pH of the solution was adjusted with a small volume (1-6 μl) of concentrated HCl or 2M NaOH solution. A total of 27 spectra (1) or 23 spectra (4) were recorded over a pH range of 0.50-12.50 (1) or 0.51-12.56 (4). The P NMR chemical shifts of the dianionic and monoanionic forms of the (thio)phosphonate groups were plotted against measured pH. All pKa values were calculated by non-linear regression analysis using the GraphPad Prism program (GraphPad Software Inc., San Diego Calif.) and the equation pH=pKa−log[(δₐ−δ)/(δ−δ_{b})] where δ is the ³¹P NMR chemical shift at varying pH, δₐ and δ_{b} the ³¹P NMR chemical shifts with titrating group in fully acidic or basic form respectively.

Pharmacology

Cell culture and transfection. Pharmacological experiments were carried out using HEK293 cells. Cells were cultured in Dulbecco's modified Eagle's medium supplemented with 10% foetal calf serum (FCS) and antibiotics (Penicillin and Streptomycin, 100 U/mL final).

Cells were transiently transfected with rat clones of group-III mGlu receptors (mGlu4, mGlu6, mGlu7 and mGlu8) by electroporation as described elsewhere (ref. 6) and plated in 96-well microplates. The high affinity glutamate transporter EAAC1 was also co-transfected with the receptor in order to avoid any influence of glutamate released by the cells in the assay medium. Since group-III mGluRs activates naturally Gi/o-proteins which modulate the adenylyl-cyclase pathway, these receptors were co-transfected with a chimeric G-protein which is recognized by these receptors but couples to the phospholipase-C pathway, thus leading to inositol phosphate (IP) production following receptor activation (ref. 7). Receptor activity was then determined by measurement of the IP production.

Culture medium, FCS and other products used for cell culture were purchased from GIBCO-BRL-Life Technologies, Inc. (Cergy Pontoise, France). Glutamate-pyruvate transaminase (GPT) was purchased from Roche (Basel, Switzerland). ³H-myoinositol (16 Ci/mmol) was purchased from Amersham (Saclay, France).

Functional assay. Inositol phosphate determination experiments in 96-well microplates were performed as already described (ref. 15). Briefly, 6 h following transfection, cell medium was removed and replaced by fresh medium devoid of glutamate, not complemented with FCS, and that contained ³H-myoinositol. Cells were incubated overnight in this medium. The following day, cells were rinsed and ambient glutamate degraded by incubation in presence of GPT. Cells were stimulated by agonist for 30 min then the medium was removed and cells incubated for 1 h with cold 0.1 M formic acid which induced cell lysis. The ³H-IP produced following receptor stimulation were recovered by ion exchange chromatography using a Dowex resin (Biorad). IP kept by the resin were then eluted by a 4 M formate solution (pH4.4) and collected in a 96-wells sample plate. Samples were then mixed with liquid scintillator (Perkin Elmer). In order to minimize well to well variability due to difference in cell density, the radioactivity remaining in the membranes which is proportional to the quantity of cells in each well was used to normalize the IP produced. Membranes were solubilized with a solution of NaOH (0.1 M) containing 10% of Triton X100 (Sigma), the resulting solution was then collected in a 96-well sample plate and mixed with liquid scintillator. Radioactivity was counted using a Wallac 1450 Microbeta stintillation and luminescence counter (Perkin Elmer). Results are expressed as the ratio between IP and the total radioactivity corresponding to IP plus membrane. All points are realized in triplicate. The dose-response curves were fitted using the GraphPad Prism program and the following equation: y=[(ymaxymin)/(1+(x/EC₅₀)n)]+ymin where EC₅₀ is the concentration of the compound necessary to obtain the half maximal effect and n is the Hill coefficient.

### Synthesis of Compounds of Formula (I) According to the Following Synthetic Schemes 3-5.

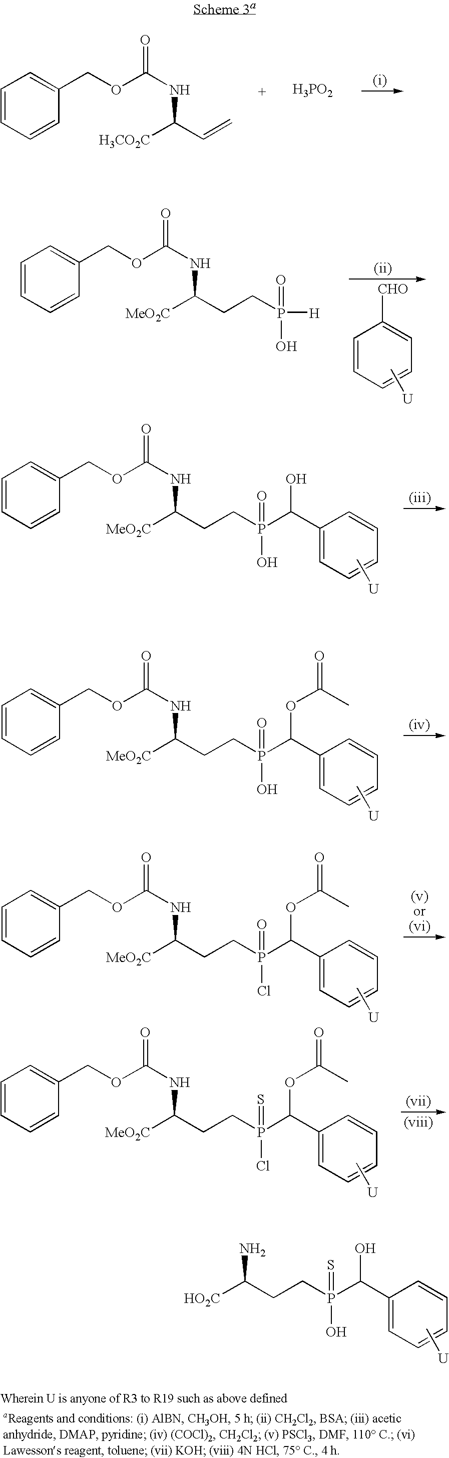

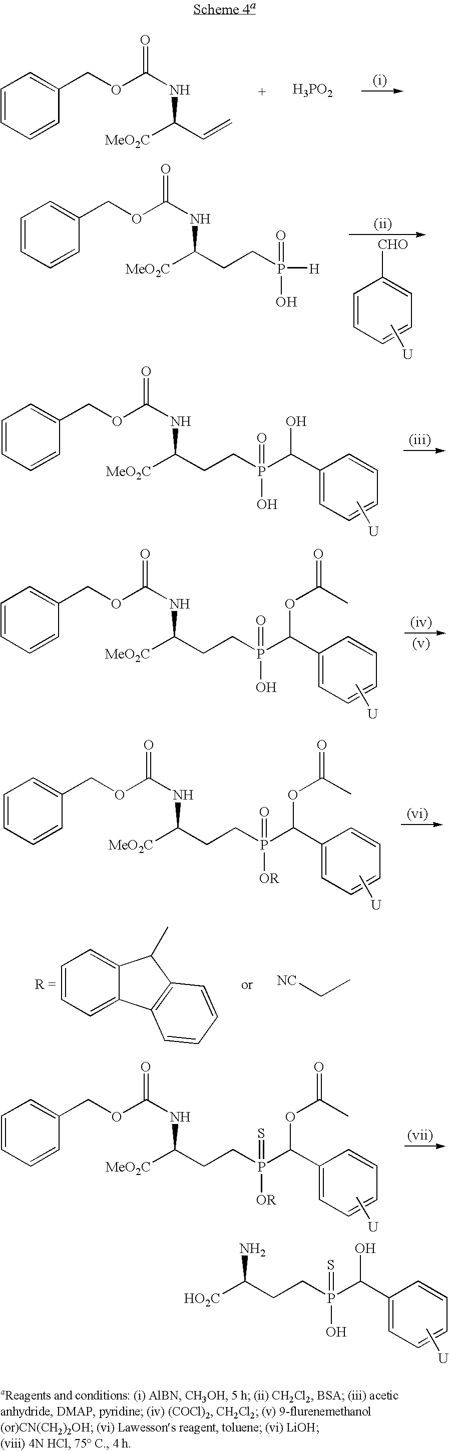

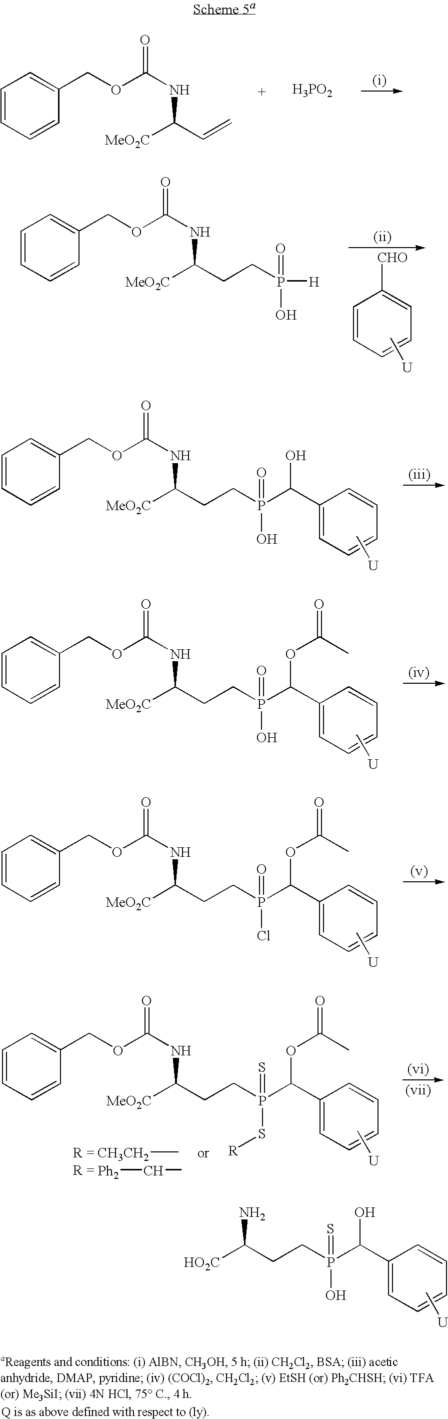

### Synthesis of Compounds of Formula (Iy) According to Synthetic Scheme 6.

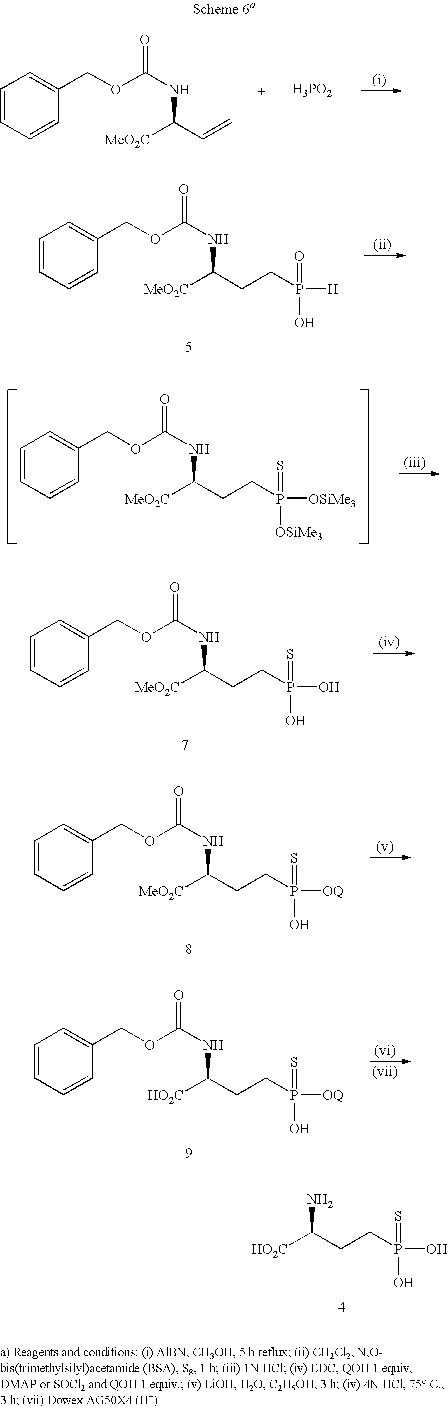

### Synthesis of Compounds of Formula (Iz) may be Prepared According to Synthetic Schemes 7-8.

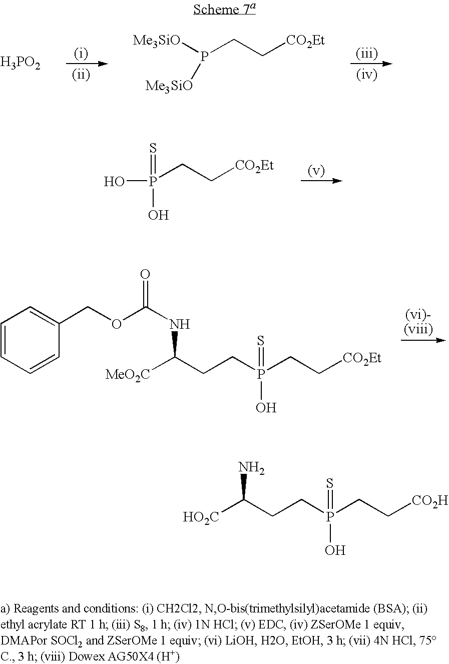

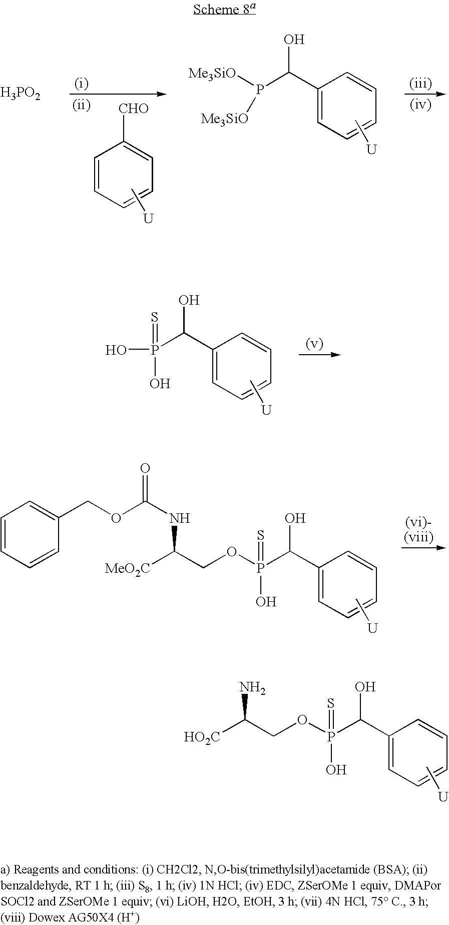

### REFERENCES

(1) Froestl, W.; Mickel, S. J.; Hall, R. G.; von Sprecher, G.; Strut), D.; Baumann, P. A.; Brugger, F.; Gentsch, C.; Jaekel, J.; Olpe, H. R.; Rills, G.; Vassout, A.; Waldmeier, P. C.; Bittiger, H. Phosphinic acid analogues of GABA. 1. New potent and selective GABA_{B} agonists. J. Med. Chem. 1995, 38, 3297-3312.
(2) Zeng, B.; Wong, K. K.; Pompliano, D. L.; Reddy, S.; Tanner, M. E. A phosphinate inhibitor of the meso-diaminopimelic acid-adding enzyme (MurE) of peptidoglycan biosynthesis. J. Org. Chem. 1998, 63, 10081-10085.
(3) Bartley, D. M.; Coward, J. K. A stereoselective synthesis of phosphinic acid phosphapeptides
(4) Albouy, D.; Brun, A.; Munoz, A.; Etemad-Moghadam, G. New (α-hydroxyalkyl)phosphorus amphiphiles: synthesis and dissociation constants. J. Org. Chem. 1998, 63, 7223-7230.
(5) SPARC Performs Automated Reasoning in Chemistry. http://ibmlc2.chem.uga.edu/sparc/.
(6) Hilal, S. H.; Karickhoff, S. W.; Carreira, L. A. Estimation of microscopic, zwitterionic ionization constants, isoelectric point and molecular speciation of organic compounds. Talanta 1999, 50, 827840.
(7) Kelley, J. L.; McLean, E. W.; Crouch, R. C.; Averett, D. R.; Tuttle, J. V. [[(Guaninylalkyl)phosphinico]methyl]phosphonic acids. Multisubstrate analog inhibitors of human erythrocyte purine nucleoside phosphorylase. J. Med. Chem. 1995, 38, 1005-1014.
(8) Stirtan, W. G.; Withers, S. G. Phosphonate and -fluorophosphonate analogue probes of the ionization state of pyridoxal 5′-phosphate (PLP) in glycogen phosphorylase. Biochemistry 1996, 35, 1505715064.
(9) Castellino, S.; Leo, G. C.; Sammons, R. D.; Sikorski, J. A. Phosphorus-31, nitrogen-15, and carbon13 and NMR of glyphosate: comparison of pH titrations to the herbicidal dead-end complex with 5enolpyruvoylshikimate-3-phosphate synthase. Biochemistry 1989, 28, 3856-3868.
(10) Gomeza, J.; Mary, S.; Brabet, I.; Parmentier, M.-L.; Restituito, S.; Bockaert, J.; Pin, J.-P. Coupling of mGluR2 and mGluR4 to Gα15, Gα16 and chimeric Gαq/i proteins: characterization of new antagonists. Mol. Pharmacol. 1996, 50, 923-930.
(11) Conklin, B. R.; Farfel, Z.; Lustig, K. D.; Julius, D.; Bourne, H. R. Substitution of three amino acids switches receptor specificity of Gqα to that of Giα. Nature 1993, 363, 274-276.
(12) Kunishima, N.; Shimada, Y.; Tsuji, Y.; Sato, T.; Yamamoto, M.; Kumasaka, T.; Nakanishi, S.; Jingami, H.; Morikawa, K. Structural basis of glutamate recognition by a dimeric metabotropic glutamate receptor. Nature 2000, 407, 971-977.
(13) Muto, T.; Tsuchiya, D.; Morikawa, K.; Jingami, H. Structures of the extracellular regions of the group II/III metabotropic glutamate receptors. Proc Nat'l Acad Sci USA. 2007, 104, 3759-3764.
(14) Bertrand, H.-O.; Bessis, A.-S.; Pin, J.-P.; Acher, F. C. Common and selective molecular determinants involved in metabotropic glutamate receptor agonist activity. J. Med. Chem. 2002, 45, 3171-3183.
(15) Goudet, C.; Gaven, F.; Kniazeff, J.; Vol, C.; Liu, J.; Cohen-Gonsaud, M.; Acher, F.; Prézeau, L.; Pin, J.-P. Heptahelical domain of metabotropic glutamate receptor 5 behaves like rhodopsin-like receptors. Proc Nat. Acad. Sci. USA 2004, 101, 378-383.

1. Thiophosphi(O)nic acid derivatives having formula (I) 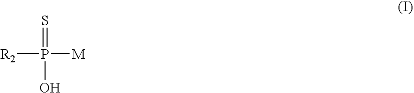
wherein
M is a [C(R₃,R₄)]ₙ₁—C(E,COOR₁,N(H,Z)) group, or an optionally substituted Ar—CH(COOR₁,N(H,Z)) group (Ar designating an aryl or an heteroaryl group), or an α, β cyclic aminoacid group such as, 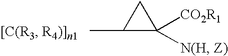
or a β,γ-cyclic aminoacid group such as 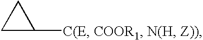
R₁ is H or R, R being an hydroxy or a carboxy protecting group, such as C₁-C₃ alkyl, Ar (being aryl or heteroaryl),
Z is H or an amino protecting group R′, such as C₁-C₃ alkyl, C₁-C₃ acyl, Boc, Fmoc, COOR, benzyl oxycarbonyl, benzyl or benzyl substituted such as defined with respect to Ar;
E is H or a C₁-C₃ alkyl, aryl, an hydrophobic group such as (CH₂)ₙ₁-alkyl, (CH₂)ₙ₁-aryl (or heteroaryl), such as a benzyl group, or a xanthyl, alkyl xanthyl or alkyl thioxanthyl group, or —(CH₂)ₙ₁-cycloalkyl, —(CH₂)ₙ—(CH₂—Ar)₂, a chromanyl group, particularly 4-methyl chromanyle, indanyle, tetrahydro naphtyl, particularly methyl-tetrahydronaphtyl; or M is OM′, wherein M′ is as above defined for M;
R₂ is selected in the group comprising:
D-CH(R₆)—C—(R₇,R₈)—
(R₁₁,R₁₂)CH—C(R₉,R₁₀)—
D-CH(OH)—
D-[C(R₁₃,R₁₄)]ₙ₃—
—C[(R₁₆,R₁₆,R₁₇)]ₙ₄—
D-CH₂—
(R₁₈)CH═C(R₁₉)—
D-(M₁)ₙ₆-CO—
D-C(R,R′)—O—
D-O— 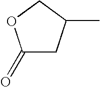
PO(OH)₂—CH₂ or (PO(OH)₂—CH₂), (COOH—CH₂)—CH₂—
with
D=H, OH, OR, (CH₂)ₙ₂OH, (CH₂)ₙ₁OR, COOH, COOR, (CH₂)ₙ₂COOH, (CH₂)ₙ₁COOR, SR, S(OR), SO₂R, NO₂, heteroaryl, C₁-C₃ alkyl, cycloalkyl, heterocycloalkyl, (CH₂)ₙ₂-alkyl, (COOH,NH₂)—(CH₂)ᵤ₁-cyclopropyl-(CH₂)ᵤ₂—, CO—NH-alkyl, Ar, (CH₂)ₙ₂—Ar, CO—NH—Ar, R being as above defined and Ar being an optionally substituted aryl or heteroaryl group,
R₃ to R₁₉, identical or different, being H, OH, OR, (CH₂)ₙ₂OH, (CH₂)ₙ₁OR, COOH, COOR, (CH₂)ₙ₂COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂, 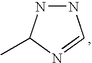
NO₂, SO₂NH₂, SO₂NHR; SR, S(O)R, SO₂R, benzyl;
one of R₁₁ or R₁₂ being COOR, COOH, (CH₂)n₂—COOH, (CH₂)n₂—COOR, PO₃H₂ the other one being such as defined for R₉ and R₁₀;
one of R₁₅, R₁₆ and R₁₇ is COOH or COOR, the others, identical or different, being such as above defined;
one of R₁₈ and R₁₉ is COOH or COOR, the other being such as above defined;
M₁ is an alkylene or arylene group;
n1=1, 2 or 3;
n2=1, 2 or 3,
n3=0, 1, 2 or 3 and
n4=1, 2 or 3;
n5=1, 2 or 3;
n6=0 or 1,
u1 and u2, identical or different=0, 1 or 2,
Ar, and alkyl groups being optionally substituted by one or several substituents on a same position or on different positions, said substituents being selected in the group comprising: OH, OR, (CH₂)ₙ₁OH, (CH₂)ₙ₁OR, COOH, COOR, (CH₂)ₙ₁COOH, (CH₂)ₙ₁COOR, C₁-C₃ alkyl, cycloalkyl, (CH₂)ₙ₁-alkyl, aryl, (CH₂)ₙ₁-aryl, halogen, CF₃, SO₃H, (CH₂)ₓPO₃H₂, with x=0, 1 or 2, B(OH)₂, 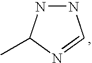
NO₂, SO₂NH₂, SO₂NHR; SR, S(O)R, SO₂R, benzyl;
R being such as above defined,

2. The thiophosphi(o)nic acid derivatives of claim 1, having formula (II) 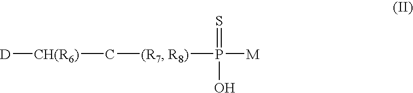
.

3. The thiophosphi(o)nic acid derivatives of claim 2, wherein D is Ar or a substituted Ar, especially a phenyl group having 1 to 5 substituents.

4. The thiophosphi(o)nic acid derivatives of claim 3, wherein the substituents are in ortho and/or meta and/or para positions and are selected in the group comprising OH, OR, (CH₂)ₙ₂OH, (CH₂)ₙ₂OR, COOH, COOR, (CH₂)ₙ₂COOH, (CH₂)ₙ₂COOR, C₁-C₃ alkyl or cycloalkyl, (CH₂)ₙ₂-alkyl, aryl, (CH₂)ₙ₂-aryl, halogen, CF₃, SO₃H, PO₃H₂, B(OH)₂ alkylamino, fluorescent group (dansyl, benzoyl dinitro 3, 5′, 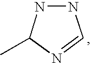
NO₂, SO₂NH₂, SO₂(NH,R)SR, S(O)R, SO₂R, OCF₃, heterocycle, heteroaryl, substituted such as above defined with respect to Ar.

5. The thiophosphi(o)nic acid derivatives of formula (III) 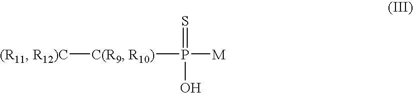
.

6. The thiophosphi(o)nic acid derivatives of claim 5, wherein one of R₁₁ or R₁₂ is COOH.

7. The thiophosphi(o)nic acid derivatives of claim 1, having formula (IV) 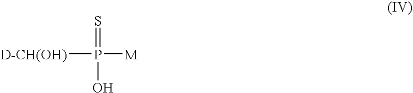
.

8. The thiophosphi(o)nic acid derivatives of claim 7, wherein D is as above defined with respect to formula II.

9. The thiophosphi(o)nic acid derivatives of claim 1, having formula (V) 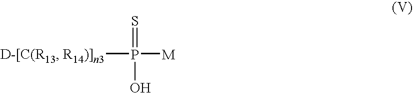
, one of R₁₃ or R₁₄ representing OH.

10. The thiophosphi(o)nic acid derivatives of claim 9, wherein D is as above defined with respect to formula II.

11. The thiophosphi(o)nic acid derivatives of claim 1, having formula (VI) 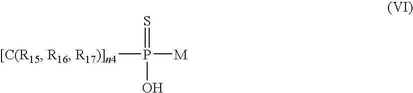
.

12. The thiophosphi(o)nic acid derivatives of claim 11, wherein, in the first group of the chain, one or two of R₁₅, R₁₆ or R₁₇ is COOH.

13. The thiophosphi(o)nic acid derivatives of claim 1, having formula (VII) 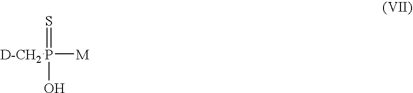
.

14. The thiophosphi(o)nic acid derivatives of claim 13, as above defined with respect to formula II.

15. The thiophosphi(o)nic acid derivatives of claim 2, wherein R₆ to R₁₀, one of R₁₁ or R₁₂, on of R₁₃ or R₁₄, one or two of R₁₅, R₁₆ or R₁₇ is H, C₁-C₃ alkyl, OH, NH₂, CF₃.

16. The thiophosphi(o)nic acid derivatives of claim 1, having formula (VIII) 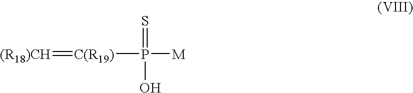
.

17. The thiophosphi(o)nic acid derivatives of claim 16, wherein R₁₈ is COOH.

18. The thiophosphi(o)nic acid derivatives of claim 16, wherein R₁₉ is H, C₁-C₃ alkyl, OH.

19. The thiophosphi(o)nic acid derivatives of claim 1, having formula LIX 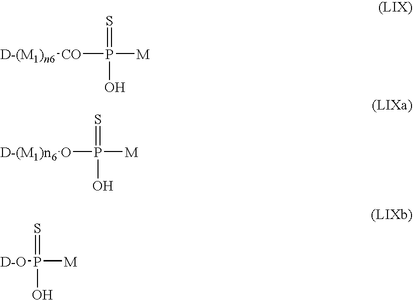
.

20. The thiophosphi(o)nic acid derivatives of claim 19, wherein either n6=0, or n6=1 and M₁ is an alkylene or an arylene group.

21. The thiophosphi(o)nic acid derivatives of claim 1, wherein M is a [C(R₃,R₄)]ₙ₁—C(E,COOR₁,N(H,Z)) group.

22. The thiophosphi(o)nic acid derivatives of claim 1, wherein M is an Ar group or a substituted arylene group, particularly a C6H4 group or a substituted C6H4 group.

23. The thiophosphi(o)nic acid derivatives of claim 1, wherein M comprises a cyclic aminoacid group, particularly an α, β cyclic aminoacid group such as 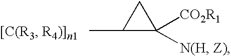
or a β,γ-cyclic aminoacid group such as

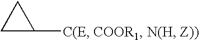

24. A process for preparing thiophosphi(o)nic acid derivatives of formula I 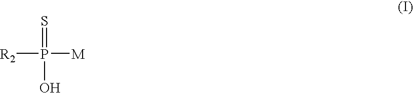
wherein the substituents are as above defined in claim 1,
comprising
according to method A):
a1) treating a derivative of formula (IX) 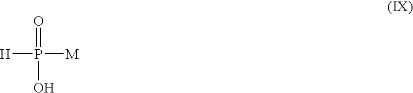
with either trimethylsilylchloride (TMSCl) and triethylamine (Et₃N), or N,O-(bis-triethylsilyl)acetamide (BSA);
a2) adding to the reaction product
one of the following derivatives having, respectively,
D-C(R₆)═C(R₇, R₈), or formula X
(R₁₁,R₁₂)C═C(R₉,R₁₀) formula XI
formula XII: 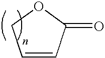
with n=1 or 2
D-CH(═O) formula XIII
D-[C(R₁₃,R₁₄)]ₙ₃—Br formula XIV
[C(R₁₅,R₁₆,R₁₇)]ₙ₄—Br formula XV
D-I formula XVI
(R₁₈)CH≡C(R₁₉) formula XVII
a3) replacing the P═O moiety by P═S moiety, by protecting the hydroxymeze group when present and the phosphonic acid before introducing the sulphur atom by the use of the Lawerson reagent or PSCl₃,
a4) performing hydrolysis in two steps, comprising 1) LiOH or KOH hydrolysis with esters; 2) deprotection under acid conditions at 60-80° C.,
a5) treating the reaction product under acidic conditions or with catalysts to obtain the final desired product;
a6) recovering the diastereoisomers or the enantiomer forms,
a7) if desired, separating diastereoisomers, when obtained, into the enantiomers.
according to method B, said process comprises
b1) treating a derivative of formula (XVIII)
(R″SiO)₂—P—H (XVIII)
wherein R″ is a C₁-C₃ alkyl
with
either a derivative of formula (X)
D-C(R₆)═C(R₇,R₈) (X)
or with a derivative of formula (XI)
(R₁₁,R₁₂)C═C(R₉,R₁₀) (XI)
wherein one of R₉ or R₁₀ is COOalk, alk being a C₁-C₃ alkyl
b2) treating the condensation product with a dibromo derivative of formula (XIX)
Br—[C(R₃,R₄)]ₙ₁—Br (XIX)
under reflux conditions; and adding HC(Oalk)₃
wherein alk is a C₁-C₃ alkyl
b3) treating the condensation product with a derivative of formula (XX)
NH(Z)-CH(CO₂R)₂ (XX)
in the presence of K₂CO₃, BuO₄NBr, under reflux conditions;
b4) replacing the P═O moiety by P═S moiety, by protecting the hydroxymeze group when present and the phosphonic acid before introducing the sulphur atom by the use of the Lawerson reagent or PSCl₃,
b5) performing hydrolysis in two steps, comprising 1) LiOH or KOH hydrolysis with esters; 2) deprotection under acid conditions at 60-80° C.,
b6) treating the condensation product under acidic conditions or with catalyst to obtain the final desired product;
b7) recovering the diastereoisomers or the enantiomer forms, and
b8) if desired, separating diastereoisomers, when obtained, into the enantiomers.
alternatively, the reaction product obtained at step b1) is reacted, according to step b2i), with a derivative of formula (XXI)
[(R₃,R₄)C]ₙ₁═C(COOR₁,NH(Z)) (XXI)
and, according to step b3i), the reaction product is treated under acidic conditions to give the final desired product.
according to method C, said process comprises
c1) reacting, as defined in step a1), a derivative of formula (XXII) 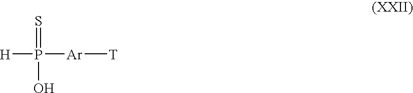
wherein Ar is as above defined and preferably an optionally substituted C₆H₄ group and T represents a C₁-C₃ alkyl group
c2) carrying out reaction step a2) by using one of the derivatives of formula (X) to (XVII)
c3) treating the reaction product with NBS, AiBN to have a bromo derivative with Ar substituted by T′-Br, with T′=CH₂
c4) reacting the bromo derivative thus obtained with (CH)₆N₄ in an organic solvent, then AcOH/H₂O to obtain a cetone derivative with Ar substituted by —C═O,
c5) treating cetone derivatives with KCN, NH₄C₁ and NH₄OH to obtain aminocyano derivatives, with Ar substituted by —C(CN,NH₂)
c6) replacing the P═O moiety by P═S moiety, by protecting the hydroxymeze group when present and the phosphonic acid before introducing the sulphur atom by the use of the Lawerson reagent or PSCl₃,
c7) performing hydrolysis in two steps, comprising 1) LiOH or KOH hydrolysis with esters; 2) deprotection under acid conditions at 60-80° C.,
c8) treating under acidic conditions to obtain derivatives with Ar substituted by —C(COOR,NH₂), and
c9) treating with catalysts to obtain the final desired product.
according to method D, for preparing compounds of formula Iy according to 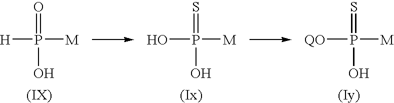
Wherein Q is D-C(R,R′)-ou D-
d1) treating a derivative of formula (IX) with N,O-(bis-triethylsilyl)acetamide (BSA) and sulfur powder
d2) hydrolysing with 1N HCl to afford thiophosphonate (Ix)
d3) reacting thiophosphonate (Ix) with EDC (N-(3-Dimethylaminopropyl)-N′-ethylcarbodiimide), alcohol QOH in DMF or reacting thiophosphonate (Ix) with SOCl₂ at 0° C. and then with 1 equivalent of alcohol Q₂OH
d4) treating the reaction product under acidic conditions or with catalysts or as C7 to obtain the final desired product (Iy).
according to method E, for preparing thiophosphonates of formula (Iz) 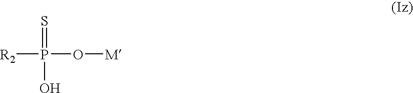
e1) Z-protected serine methyl ester O-phosphate (Z-Ser-(OMe)-O-phosphate) or homologues for example (homoserine) are treated as (Ix) in d3) (diesterification) and then as described in a3) and a4).
e2) or treating H₃PO₂ hypophorous acid as in b1) followed by reacting the condensation product as in d1) to afford (Iz) with M′=H, which is then reacted with Z-protected serine methyl ester (Z-Ser-(OMe)-OH) or homologues as in d3) and d4).

25. The process of claim 24, wherein
in method A, according to a preferred embodiment
the use of derivatives of formula (X)
D-CH(R₆)═C(R₇,R₈) (X)
with derivatives of formula (IX) results, in step a2), in intermediate derivatives of formula (XXIII) 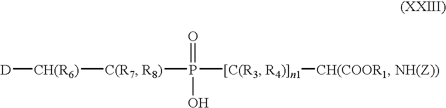
and, in step a5), in a final product of formula (XXIV) 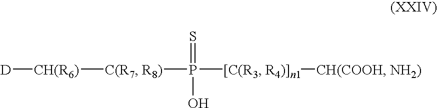
the use of derivatives of formula (XI) or formula (XII) 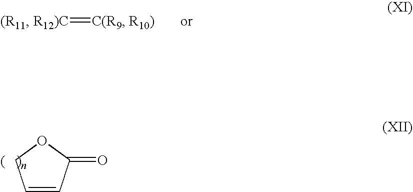
results, in step a2), in intermediate derivatives of formula (XXV) 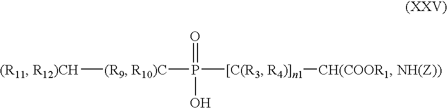
and, in step a5), in a final product of formula (XXVI) 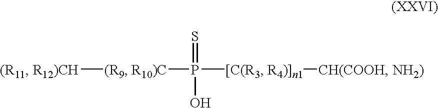
the use of derivatives of formula (XIII)
D-CH(═O) (XIII)
results, in step a2), in intermediate derivatives of formula (XXVII) 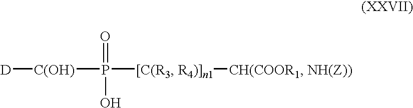
and, in step a5), in a final product of formula (XXVIII) 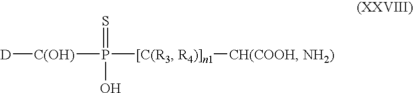
the use of derivatives of formula (XIV)
D-[C(R₁₃,R₁₄)]ₙ₃—Br (XIV)
results, in step a2), in intermediate derivatives of formula (XXIX) 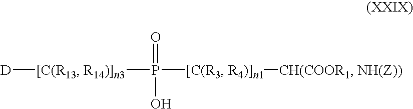
and, in step a5), in a final product of formula (XXX) 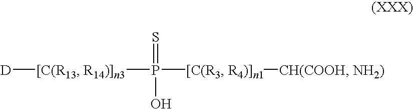
the use of derivatives of formula (XV)
[C(R₁₆,R₁₆,R₁₇)]ₙ₄—Br (XV)
results, in step a3), in intermediate derivatives of formula (XXXI) 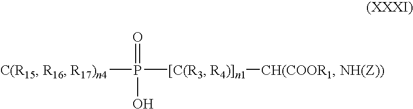
and, in step a5), in a final product of formula (XXXII) 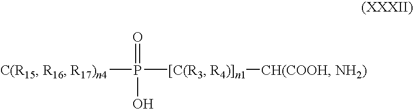
the use of derivatives of formula (XVI)
D-I (XVI)
results, in step a2), in intermediate derivatives of formula (XXXIII) 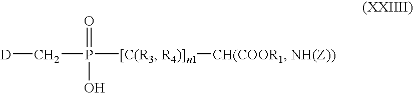
and, in step a5), in a final product of formula (XXXIV) 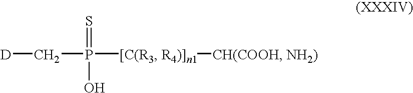
the use of derivatives of formula (XVII)
(R₁₈)C≡C(R₁₉) (XVII)
results, in step a2), in intermediate derivatives of formula (XXXV) 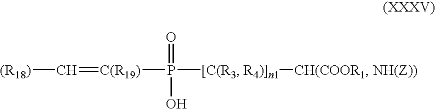
and, in step a5), in a final product of formula (XXXVI) 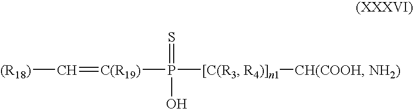
the use of derivatives of formula (LIX) 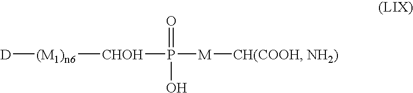
wherein M₁ is as above defined with respect to M and results by oxidation in a product of formula (LXI)

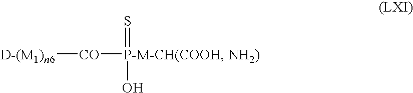

26. The method of claim 24, wherein
in method B,
the use, with derivatives of formula (XVIII), of derivatives of formula (X)
D-CH(R₆)—C(R₇,R₈) (X)
results, in step b1), in intermediate derivatives of formula (XXXVII)
D-CH(R₆)—C(R₇,R₈)—P—(OSiR″)₂ (XXXVII)
in step b2), in intermediate derivatives of formula (XXXVIII) 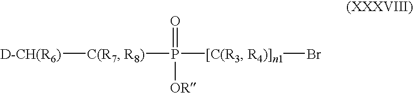
in step b5), in intermediate derivatives of formula (XXXIX) 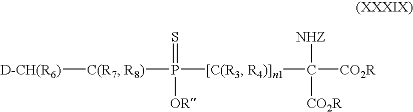
and, in step b6), in a final product of formula (XXXX) 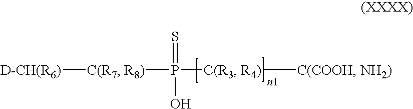
the use, with derivatives of formula (XVIII), of derivatives of formula (XI)
(R₁₁,R₁₂)C═C(R₅,R₁₀) (XI)
results, in step b1), in intermediate derivatives of formula (XXXXI)
(R₁₁,R₁₂)CH—C(R₉,R₁₀)—P—(OSiR″)₂ (XXXXI)
in step b2), in intermediate derivatives of formula (XXXXII) 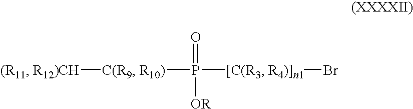
in step b5), in intermediate derivatives of formula (XXXXIII) 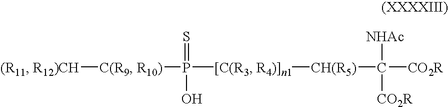
in step b6), in final products of formula (XXXXIV) 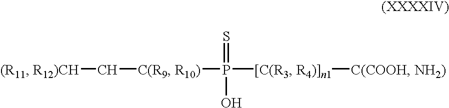
or, alternatively,
the use with derivatives of formula (XXXXI) obtained according to step b1) is reacted with a derivative of formula (XXXXV)
[(R₃,R₄)C]ₙ₁═C(COOR,NH(Z) (XXXXV)
giving intermediate derivatives of formula (XXXXVI) 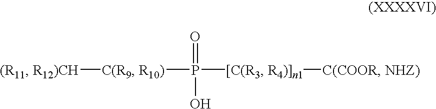
wherein the OH— group is then protected, the P═O moiety is replaced by a P═S moiety, the treatment under acidic conditions giving the final product of formula (XXXXVII)

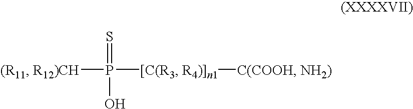

27. The process of claim 24, wherein
in method C,
the use, of a derivative of formula (XXII), 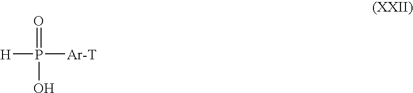
with a derivative of
D-C(R₆)═C(R₇,R₈), or formula X
(R₁₁,R₁₂)C═C(R₉,R₁₀) formula XI
formula XII: 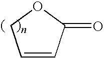
D-CH(═O) formula XIII
D-[C(R₁₃,R₁₄)]ₙ₃−Br formula XIV
[C(R₁₅,R₁₆,R₁₇)]ₙ₄—Br formula XV
D-I formula XVI
(R₁₈)C≡C(R₁₉) formula XVII
results in intermediate derivatives respectively having formulae (XXXXVIII) to (LIV)

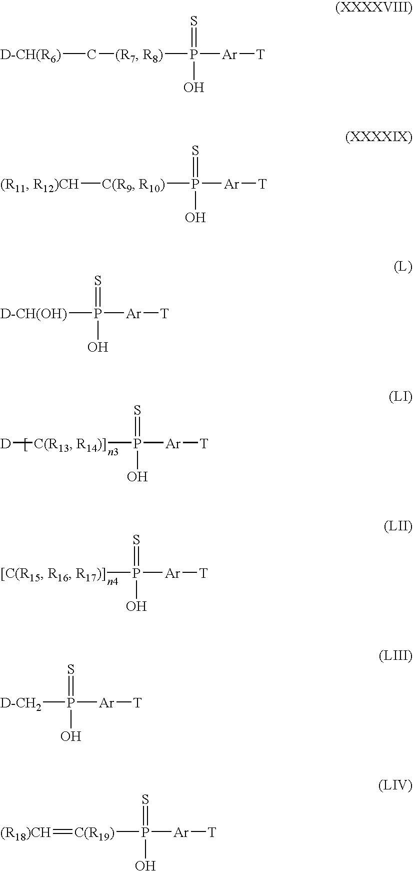

28. The process of claim 24, wherein
in method A, the derivatives of formula IX 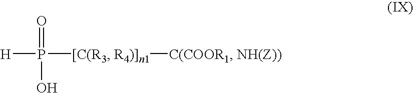
are advantageously obtained by reacting thiophosphi(o)nic acid of formula (LV) 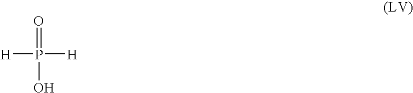
with a derivative of formula (LVI)
(R₃,R₄)ₙ₁C═CH—C(E,COOR₁,NH(Z)) (LVI)
preferably Z-vinyl-glyOMe or a derivative thereof with E different from H,
the reaction being advantageously carried out in the presence of AIBN by heating above 50° C.-100° C., preferably at about 80° C.

29. The process of claim 24, wherein
in method B, the derivatives of formula (XVIII)
(R″SiO)₂—P—H (XVIII)
are obtained by reacting an thiophosphi(o)nic acid ammonium salt of formula (LVII) 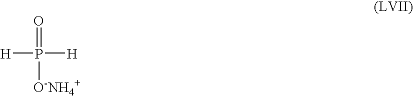
with hexamethyl disilazane of formula (LVIII)
(alk₃Si)—NH (LVIII)
the reaction being carried under an inert gas, by heating above 100° C., particularly at about 120° C.,
or by reacting hypophosphorous acid with N,O-(bis-triethylsilyl)acetamide (BSA) at room temperature.

30. The method of claim 24, wherein
in method C, the derivatives of formula (XXII) 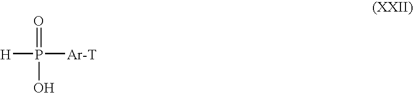
are advantageously obtained by reacting a mixture of H₃PO₂, Ar—NH₂, Ar—Br and a catalyst Pd(0) Ln (Ln=n ligands).

31. Thiophosphi(O)nic acid derivatives which are intermediates in the process of claim 24.

32. Pharmaceutical compositions comprising an effective amount of at least one of the thiophosphi(o)nic acid derivatives according to claim 1 in combination with a pharmaceutically acceptable carrier.

33. The pharmaceutical compositions according to claim 32, which are under a form suitable for an administration by the oral route, such as tablets, pills or capsules.

34. The pharmaceutical compositions of claim 33, comprising 1 to 100 mg of active ingredient per dose unit.

35. The pharmaceutical compositions according to claim 32, which are under a form suitable for an administration by injection, such as injectable solutions for the intravenous, subcutaneous or intramuscular route.

36. The pharmaceutical compositions of claim 35, comprising 1 to 30 mg of active ingredient per dose unit.

37. The pharmaceutical compositions of claim 32 for treating convulsions, pain, drug addiction, anxiety disorders and neurodegenerative diseases.

38. Use of at least one of the thiophosphi(o)nic acid derivatives of claim 1 for preparing a drug for treating brain disorders.

39. A method of treatment of brain disorders, comprising administering to a patient in need thereof an effective amount of a thiophosphi(o)nic acid derivative according to claim 1.
